SEQUENCE LISTING (13 sequences)

SEQ ID NO: 1 - protein, 317 aa, Homo sapiens
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 2 - DNA, 2201 bp, Homo sapiens
  source 1..2201: mol_type = genomic DNA
gcccgctcgcccgcgcgccccaggacccaaagccgggctccaagtcggcgccccacgtcgaggctccgccgcagcctccggagttggccgcagacaagaaggggagggagcgggagagggaggagagctccgaagcgagagggccgagcgccatgcgccgcgccagcagagactacaccaagtacctgcgtggctcggaggagatgggcggcggccccggagccccgcacgagggccccctgcacgccccgccgccgcctgcgccgcaccagccccctgccgcctcccgctccatgttcgtggccctcctggggctggggctgggccaggttgtctgcagcgtcgccctgttcttctatttcagagcgcagatggatcctaatagaatatcagaagatggcactcactgcatttatagaattttgagactccatgaaaatgcagattttcaagacacaactctggagagtcaagatacaaaattaatacctgattcatgtaggagaattaaacaggcctttcaaggagctgtgcaaaaggaattacaacatatcgttggatcacagcacatcagagcagagaaagcgatggtggatggctcatggttagatctggccaagaggagcaagcttgaagctcagccttttgctcatctcactattaatgccaccgacatcccatctggttcccataaagtgagtctgtcctcttggtaccatgatcggggttgggccaagatctccaacatgacttttagcaatggaaaactaatagttaatcaggatggcttttattacctgtatgccaacatttgctttcgacatcatgaaacttcaggagacctagctacagagtatcttcaactaatggtgtacgtcactaaaaccagcatcaaaatcccaagttctcataccctgatgaaaggaggaagcaccaagtattggtcagggaattctgaattccatttttattccataaacgttggtggattttttaagttacggtctggagaggaaatcagcatcgaggtctccaacccctccttactggatccggatcaggatgcaacatactttggggcttttaaagttcgagatatagattgagccccagtttttggagtgttatgtatttcctggatgtttggaaacattttttaaaacaagccaagaaagatgtatataggtgtgtgagactactaagaggcatggccccaacggtacacgactcagtatccatgctcttgaccttgtagagaacacgcgtatttacagccagtgggagatgttagactcatggtgtgttacacaatggtttttaaattttgtaatgaattcctagaattaaaccagattggagcaattacggggtgaccttatgagaaactgcatgtgggctatgggaggggttggtccctggtcatgtgccccttcgcagctgaagtggagagggtgtcatctagcgcaattgaaggatcatctgaaggggcaaattcttttgaattgttacatcatgctggaacctgcaaaaaatactttttctaatgaggagagaaaatatatgtatttttatataatatctaaagttatatttcagatgtaatgttttctttgcaaagtattgtaaattatatttgtgctatagtatttgattcaaaatatttaaaaatgtcttgctgttgacatatttaatgttttaaatgtacagacatatttaactggtgcactttgtaaattccctggggaaaacttgcagctaaggaggggaaaaaaatgttgtttcctaatatcaaatgcagtatatttcttcgttctttttaagttaatagattttttcagacttgtcaagcctgtgcaaaaaaattaaaatggatgccttgaataataagcaggatgttggccaccaggtgcctttcaaatttagaaactaattgactttagaaagctgacattgccaaaaaggatacataatgggccactgaaatctgtcaagagtagttatataattgttgaacaggtgtttttccacaagtgccgcaaattgtaccttttttgttttttcaaaatagaaaagttattagtggtttatcagcaaaaaagtccaattttaatttagtaaatgttatcttatactgtacaataaaaacattgcctttgaatgttaattttttggtacaaaaataaatttatatgaaaacctgc

SEQ ID NO: 3 - protein, 5 aa, synthetic construct
SYAMS

SEQ ID NO: 4 - protein, 16 aa, synthetic construct
GITGSGGSTYYADSVK

SEQ ID NO: 5 - protein, 13 aa, synthetic construct
DPGTTVIMSWFDP

SEQ ID NO: 6 - protein, 12 aa, synthetic construct
RASQSVRGRYLA

SEQ ID NO: 7 - protein, 7 aa, synthetic construct
GASSRAT

SEQ ID NO: 8 - protein, 9 aa, synthetic construct
QQYGSSPRT

SEQ ID NO: 9 - protein, 122 aa, synthetic construct
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSS

SEQ ID NO: 10 - protein, 108 aa, synthetic construct
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIK

SEQ ID NO: 11 - protein, 448 aa, synthetic construct
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 215 aa, synthetic construct
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 446 aa, synthetic construct
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSP

(19) United States
(12) Patent Application Publication
(10) Pub. No.: US 2026/0001957 A1
(43) Pub. Date: Jan. 1, 2026

(54) TREATMENT OF EROSIVE HAND OSTEOARTHRITIS

(71) Applicants: AMGEN INC., Thousand Oaks, CA (US); GHENT UNIVERSITY, Ghent (BE)

(72) Inventors: Robert Kees Stad, Rich-Rotkreuz (CH); Ruth Wittoek, Ghent (BE); August Verbruggen, Ghent (BE); Dirk Elewaut, Ghent (BE)

(21) Appl. No.: 18/993,033

(22) PCT Filed: Jul. 10, 2023
(86) PCT No.: PCT/US2023/069905

§ 371 Date: Jan. 10, 2025

Related U.S. Application Data
(60) Provisional application No. 63/359,948, filed on Jul. 11, 2022.

(51) Int. Cl. C07K 16/28; A61K 39/00; A61P 19/02

(52) U.S. Cl. CPC C07K 16/2875; A61P 19/02; A61K 2039/505; A61K 2039/545

(57) ABSTRACT

Provided herein are methods of treating erosive hand osteoarthritis (OA) in a subject in need thereof, methods of reducing radiographic erosive progression, joint space narrowing, cartilage degradation, and/or bone formation in a subject in need thereof (e.g., a subject suffering from erosive hand OA), methods of inhibiting the development of new erosive joints in a subject in need thereof (e.g., a subject suffering from erosive hand OA), and methods of reducing pain in a subject with erosive hand OA. Also provided herein are uses of denosumab in the manufacture of a medicament adapted for use in a method described herein, as well as pharmaceutical compositions comprising denosumab for use in a method described herein.

### CROSS-REFERENCE TO RELATED APPLICATIONS

This application claims the benefit of U.S. Provisional Patent Application No. 63/359,948, filed Jul. 11, 2022, which is hereby incorporated by reference in their entiretics

### FIELD OF THE INVENTION

This application relates to methods of treating erosive hand osteoarthritis.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: text file named “58297_SeqListing.xml,” 15,822 bytes, created on Jul. 10, 2023.

### BACKGROUND

The radiographically erosive type of hand osteoarthritis (OA) affecting the interphalangeal (IP) finger joints is a highly prevalent disease (1-3) and is considered to be an inflammatory subtype of hand OA (4). It is characterized by a high burden of disease, with the majority of patients suffering from substantial pain and disability (5, 6).

Radiographic hallmarks of erosive hand OA include the resorption of articular cartilage, which usually precedes the collapse of the subchondral bone endplate and osteolytic changes in the epiphyseal subchondral bone area (11-13). Articular cartilage resorption is caused by chondrocytes in degenerative cartilage undergoing hypertrophic differentiation. Hypertrophic chondrocytes and osteoclasts both release Receptor Activator of Nuclear Factor kappa-β Ligand (RANKL), which is a key driver of osteoclast maturation and activation in physiological and pathological conditions (14-18). Articular tissue destruction is followed by reparative processes such as the remodeling of the subchondral bone plate and the formation of bony nodules at the margins of the affected interphalangeal (IP) joints. Erosive features and signs of remodeling can occur simultaneously in the same patient since the disease affects the proximal and distal IP joints in an unpredictable order, causing the disease to be active until all joints progress to the terminal phase of remodeling, a process that may take several decades to complete (19).

Existing therapies for erosive hand OA only alleviate subjective complaints and do not attenuate or arrest structural damage at the IP finger joints that contributes to functional limitations and eventually results in considerable disability and chronic pain (2, 7, 8-10). Thus, there is a need in the art for alternative methods of treating erosive hand OA.

### SUMMARY

Presented herein are data from a monocentric clinical trial, wherein 100 patients with erosive hand OA were randomized to 48 weeks of blinded treatment with (a) 60 mg of the RANKL inhibitor denosumab once every about 3 months or (b) placebo, followed by an open-label extension (OLE) period through week 96. 51 and 49 patients received denosumab and placebo, respectively. A primary endpoint of the trial was the change in total Ghent University Scoring System (GUSS™) score at week 24. The GUSS™ scoring system is composed of 3 subdomains: subchondral plate; subchondral bone; and joint space. Specific features referring to the underlying pathology of the disease are scored on a numerical scale from 0 to 100, with increments of 10, where the maximum score refers to a normal or completely restored (i.e., non-erosive) joint. Secondary trial endpoints included the number of new erosive joints at week 48, as well as total GUSS™ change at week 48. Exploratory clinical and safety outcomes were also assessed. At week 24, the total change in GUSS™ was found to be statistically higher in denosumab-treated patients compared to those administered placebo (mean change GUSS™=8.9 (95% CI: 1.0-16.9; p=0.024)), a change that further increased at week 48 (AGUSS™=14.3 (95% CI: 4.6-24.0; p=0.003)). Additionally, the development of new erosive joints was significantly reduced in denosumab-treated patients (7.0%) compared to placebo-treated patients (1.9%) at week 48 (OR=0.23 (95% CI: 0.10 to 0.50); p<0.001). During OLE, both groups continued remodeling, and clinical endpoints (e.g., pain and function) significantly changed after 96 weeks of treatment. Equal numbers of adverse events occurred in both groups. Taken together, these results indicate that denosumab has structure-modifying effects in erosive hand OA compared to placebo, with significantly less erosive progression occurring after 24 weeks of denosumab treatment. This treatment effect was enhanced after 48 weeks, with symptomatic improvement seen during longer treatment periods (e.g., after 96 weeks of treatment).

Disclosed herein is a method of treating erosive hand osteoarthritis (OA) in a subject in need thereof. In some embodiments, the method comprises administering to the subject denosumab in an amount equivalent to about 45 mg to about 60 mg (e.g., about 45 mg to less than 60 mg; about 45 mg to about 55 mg; about 45 mg to about 50 mg; about 45 mg; about 50 mg; about 55 mg; about 60 mg) denosumab administered once every about 3 months. In some embodiments, the method comprises administering to the subject denosumab in an amount equivalent to about 45 mg denosumab administered once every about 3 months. In some embodiments, the method comprises administering to the subject denosumab in an amount equivalent to about 50 mg denosumab administered once every about 3 months. In some embodiments, the method comprises administering to the subject denosumab in an amount equivalent to about 55 mg denosumab administered once every about 3 months. In some embodiments, the method comprises administering to the subject denosumab in an amount equivalent to about 60 mg denosumab administered once every about 3 months.

In some embodiments, the method comprises administering to the subject about 45 mg denosumab once every about 3 months. In some embodiments, the method comprises administering to the subject about 50 mg denosumab once every about 3 months. In some embodiments, the method comprises administering to the subject about 55 mg denosumab once every about 3 months. In some embodiments, the method comprises administering to the subject about 60 mg denosumab once every about 3 months.

In some embodiments, the amount of denosumab provides:
a. a mean serum concentration of denosumab substantially similar to that achieved by administering 60 mg denosumab once every about 3 months; and/or
b. a mean plasma area under the curve (AUC_{0-∞}) substantially similar to that achieved by administering 60 mg denosumab once every about 3 months; and/or
c. a difference between the mean plasma Cₘₐₓ at steady state and a mean plasma Cₘᵢₙ at steady state substantially similar to that achieved by administering 60 mg denosumab once every about 3 months; and/or
d. a mean plasma Cₘₐₓ at steady state substantially similar to that achieved by administering denosumab 60 mg once every about 3 months.

In some embodiments, the amount of denosumab provides:
a. a mean serum concentration of denosumab substantially similar to that achieved by administering 55 mg denosumab once every about 3 months; and/or
b. a mean plasma area under the curve (AUC_{0-∞}) substantially similar to that achieved by administering 55 mg denosumab once every about 3 months; and/or
c. a difference between the mean plasma Cₘₐₓ at steady state and a mean plasma Cₘᵢₙ at steady state substantially similar to that achieved by administering 55 mg denosumab once every about 3 months; and/or
d. a mean plasma Cₘₐₓ at steady state substantially similar to that achieved by administering denosumab 55 mg once every about 3 months.

In some embodiments, the amount of denosumab provides:
a. a mean serum concentration of denosumab substantially similar to that achieved by administering 50 mg denosumab once every about 3 months; and/or
b. a mean plasma area under the curve (AUC_{0-∞}) substantially similar to that achieved by administering 50 mg denosumab once every about 3 months; and/or
c. a difference between the mean plasma Cₘₐₓ at steady state and a mean plasma Cₘᵢₙ at steady state substantially similar to that achieved by administering 50 mg denosumab once every about 3 months; and/or
d. a mean plasma Cₘₐₓ at steady state substantially similar to that achieved by administering denosumab 50 mg once every about 3 months.

In some embodiments, the amount of denosumab provides:
a. a mean serum concentration of denosumab substantially similar to that achieved by administering 45 mg denosumab once every about 3 months; and/or
b. a mean plasma area under the curve (AUC_{0-∞}) substantially similar to that achieved by administering 45 mg denosumab once every about 3 months; and/or
c. a difference between the mean plasma Cₘₐₓ at steady state and a mean plasma Cₘᵢₙ at steady state substantially similar to that achieved by administering 45 mg denosumab once every about 3 months; and/or
d. a mean plasma Cₘₐₓ at steady state substantially similar to that achieved by administering denosumab 45 mg once every about 3 months.

In some embodiments, the subject is administered denosumab for at least 12 weeks. In some embodiments, the subject is administered denosumab for at least 24 weeks. In some embodiments, the subject is administered denosumab for at least 48 weeks. In some embodiments, the subject is administered denosumab for at least 96 weeks.

In some embodiments, the subject is administered denosumab once every about 3 months. In some embodiments, the subject is administered denosumab once every about 3 months for at least 2 cycles. In some embodiments, the subject is administered denosumab once every about 3 months for at least 3 cycles. In some embodiments, the subject is administered denosumab once every about 3 months for at least 4 cycles. In some embodiments, the subject is administered denosumab once every about 3 months for at least 5 cycles. In some embodiments, the subject is administered denosumab once every about 3 months for at least 6 cycles. In some embodiments, the subject is administered denosumab once every about 3 months for at least 7 cycles. In some embodiments, the subject is administered denosumab once every about 3 months for at least 8 cycles.

In some embodiments, denosumab is administered to the subject by injection. In some embodiments, denosumab is administered to the subject by subcutaneous injection. In some embodiments, denosumab is administered to the subject by injection into the upper arm, upper thigh, or abdomen of the subject. In some embodiments, denosumab is administered to the subject by subcutaneous injection into the upper arm, upper thigh, or abdomen of the subject.

In some embodiments, the method further comprises administering to the subject at least one therapeutic agent chosen from analgesics and non-steroidal anti-inflammatory drugs. In some embodiments, the method further comprises administering to the subject a stable dose of at least one therapeutic agent chosen from analgesics and non-steroidal anti-inflammatory drugs. In some embodiments, the method further comprises administering to the subject daily supplements of oral calcium and vitamin D₃. In some embodiments, the method further comprises administering to the subject daily supplements of oral calcium (1000 mg elemental calcium) and vitamin D₃ (880 international units).

In some embodiments, the administration reduces radiographic erosive progression in the subject. In some embodiments, the administration reduces joint space narrowing in the subject. In some embodiments, the administration reduces cartilage degradation in the subject. In some embodiments, the administration reduces bone formation in the subject. In some embodiments, the administration reduces the number of new erosive joints in the subject. In some embodiments, the administration reduces the number of new erosive joints in the subject at 48 weeks. In some embodiments, the administration inhibits the development of new erosive joints in the subject. In some embodiments, the administration reduces the number of ‘S/J’ IP joints that develop ‘E’ phases in the subject. In some embodiments, the administration reduces the number of ‘S/J’ IP joints that develop ‘E’ phases in the subject at 48 weeks. In some embodiments, the administration inhibits the development of ‘E’ phases in ‘S/J’ IP joints in the subject.

In some embodiments, the administration improves erosive IP finger joint remodeling in the subject. In some embodiments, the administration improves erosive IP finger joint remodeling in the subject at one year. In some embodiments, the administration improves erosive IP finger joint remodeling in the subject at two years. In some embodiments, the administration increases total GUSS™ in the subject. In some embodiments, the administration increases total GUSS™ in the subject at 12 weeks. In some embodiments, the administration increases total GUSS™ in the subject at 48 weeks.

In some embodiments, the administration reduces pain in the subject. In some embodiments, the administration reduces pain in the subject at 96 weeks. In some embodiments, pain reduction is assessed by numeric rating scale (NRS) pain.

In some embodiments, the administration improves joint function in the subject. In some embodiments, the administration improves joint function in the subject as assessed by the Australian-Canadian Hand Osteoarthritis Index (AUSCAN). In some embodiments, the administration improves joint function in the subject as assessed by the Functional Index for Hand Osteoarthritis (FIHOA).

In some embodiments, the subject does not exhibit a dose limiting toxicity (DLT) during denosumab administration. In some embodiments, the subject does not exhibit any grade 3 or grade 4 adverse events associated with denosumab during denosumab administration. In some embodiments, the subject is not hypocalcemic during denosumab administration.

In some embodiments, the subject exhibits at least one sign of clinical or sonographic inflammation prior to denosumab administration. In some embodiments, the subject exhibits at least one sign of clinical or sonographic inflammation in at least one interphalangeal finger joint prior to denosumab administration. In some embodiments, the subject exhibits at least one interphalangeal finger joint in the J or E phase with at least one inflammatory sign prior to denosumab administration. In some embodiments, the subject exhibits at least one interphalangeal finger joint in the J or E phase with palpable swelling prior to denosumab administration.

In some embodiments, the subject is at least about 18 years old. In some embodiments, the subject is at least about 30 years old. In some embodiments, the subject has not been diagnosed with vitamin D deficiency. In some embodiments, the subject has not been diagnosed with a chronic inflammatory rheumatic disease. In some embodiments, the subject has not been diagnosed with psoriasis. In some embodiments, the subject has not been diagnosed with cancer. In some embodiments, the subject has not been diagnosed with a chronic infectious disease.

In some embodiments, the subject has not been previously treated for erosive hand osteoarthritis. In some embodiments, the subject was previously treated for erosive hand osteoarthritis.

In some embodiments, the subject was previously treated with at least one therapeutic agent chosen from tumor necrosis factor α (TNFα) blocking agents, anti-interleukin-1α inhibitors, anti-interleukin-1β inhibitors, and combinations thereof. In some embodiments, the subject was previously treated with at least one therapeutic agent chosen from adalimumab, etanercept, lutikizumab, and combinations thereof.

Also disclosed herein is use of denosumab in the manufacture of a medicament adapted for use in a method of treating erosive hand osteoarthritis in a subject in need thereof. In some embodiments, the method comprises administering to the subject denosumab in an amount equivalent to about 45 mg to about 60 mg (e.g., about 45 mg to less than 60 mg; about 45 mg to about 55 mg; about 45 mg to about 50 mg; about 45 mg; about 50 mg; about 55 mg; about 60 mg) denosumab administered once every about 3 months. In some embodiments, the method comprises administering to the subject denosumab in an amount equivalent to about 45 mg denosumab administered once every about 3 months. In some embodiments, the method comprises administering to the subject denosumab in an amount equivalent to about 50 mg denosumab administered once every about 3 months. In some embodiments, the method comprises administering to the subject denosumab in an amount equivalent to about 55 mg denosumab administered once every about 3 months. In some embodiments, the method comprises administering to the subject denosumab in an amount equivalent to about 60 mg denosumab administered oncc every about 3 months.

In some embodiments, the method comprises administering to the subject about 45 mg denosumab once every about 3 months. In some embodiments, the method comprises administering to the subject about 50 mg denosumab once every about 3 months. In some embodiments, the method comprises administering to the subject about 55 mg denosumab once every about 3 months. In some embodiments, the method comprises administering to the subject about 60 mg denosumab once every about 3 months.

In some embodiments, the amount of denosumab provides:
a. a mean serum concentration of denosumab substantially similar to that achieved by administering 60 mg denosumab once every about 3 months; and/or
b. a mean plasma area under the curve (AUC_{0-∞}) substantially similar to that achieved by administering 60 mg denosumab once every about 3 months; and/or
c. a difference between the mean plasma Cₘₐₓ at steady state and a mean plasma Cₘᵢₙ at steady state substantially similar to that achieved by administering 60 mg denosumab once every about 3 months; and/or
d. a mean plasma Cₘₐₓ at steady state substantially similar to that achieved by administering denosumab 60 mg once every about 3 months.

In some embodiments, the amount of denosumab provides:
a. a mean serum concentration of denosumab substantially similar to that achieved by administering 55 mg denosumab once every about 3 months; and/or
b. a mean plasma area under the curve (AUC_{0-∞}) substantially similar to that achieved by administering 55 mg denosumab once every about 3 months; and/or
c. a difference between the mean plasma Cₘₐₓ at steady state and a mean plasma Cₘᵢₙ at steady state substantially similar to that achieved by administering 55 mg denosumab once every about 3 months; and/or
d. a mean plasma Cₘₐₓ at steady state substantially similar to that achieved by administering denosumab 55 mg once every about 3 months.

In some embodiments, the amount of denosumab provides:
a. a mean scrum concentration of denosumab substantially similar to that achieved by administering 50 mg denosumab once every about 3 months; and/or
b. a mean plasma area under the curve (AUC_{0-∞}) substantially similar to that achieved by administering 50 mg denosumab once every about 3 months; and/or
c. a difference between the mean plasma Cₘₐₓ at steady state and a mean plasma Cₘᵢₙ at steady state substantially similar to that achieved by administering 50 mg denosumab once every about 3 months; and/or
d. a mean plasma Cₘₐₓ at steady state substantially similar to that achieved by administering denosumab 50 mg once every about 3 months.

In some embodiments, the amount of denosumab provides:
a. a mean serum concentration of denosumab substantially similar to that achieved by administering 45 mg denosumab once every about 3 months; and/or
b. a mean plasma area under the curve (AUC_{0-∞}) substantially similar to that achieved by administering 45 mg denosumab once every about 3 months; and/or
c. a difference between the mean plasma Cₘₐₓ at steady state and a mean plasma Cₘᵢₙ at steady state substantially similar to that achieved by administering 45 mg denosumab once every about 3 months; and/or
d. a mean plasma Cₘₐₓ at steady state substantially similar to that achieved by administering denosumab 45 mg once every about 3 months.

In some embodiments, the subject is administered denosumab for at least 12 weeks. In some embodiments, the subject is administered denosumab for at least 24 weeks. In some embodiments, the subject is administered denosumab for at least 48 weeks. In some embodiments, the subject is administered denosumab for at least 96 weeks.

In some embodiments, the subject is administered denosumab once every about 3 months. In some embodiments, the subject is administered denosumab once every about 3 months for at least 2 cycles. In some embodiments, the subject is administered denosumab once every about 3 months for at least 3 cycles. In some embodiments, the subject is administered denosumab once every about 3 months for at least 4 cycles. In some embodiments, the subject is administered denosumab once every about 3 months for at least 5 cycles. In some embodiments, the subject is administered denosumab once every about 3 months for at least 6 cycles. In some embodiments, the subject is administered denosumab once every about 3 months for at least 7 cycles. In some embodiments, the subject is administered denosumab once every about 3 months for at least 8 cycles.

In some embodiments, denosumab is administered to the subject by injection. In some embodiments, denosumab is administered to the subject by subcutaneous injection. In some embodiments, denosumab is administered to the subject by injection into the upper arm, upper thigh, or abdomen of the subject. In some embodiments, denosumab is administered to the subject by subcutaneous injection into the upper arm, upper thigh, or abdomen of the subject.

In some embodiments, the method further comprises administering to the subject at least one therapeutic agent chosen from analgesics and non-steroidal anti-inflammatory drugs. In some embodiments, the method further comprises administering to the subject a stable dose of at least one therapeutic agent chosen from analgesics and non-steroidal anti-inflammatory drugs. In some embodiments, the method further comprises administering to the subject daily supplements of oral calcium and vitamin D₃. In some embodiments, the method further comprises administering to the subject daily supplements of oral calcium (1000 mg elemental calcium) and vitamin D₃ (880 international units).

In some embodiments, the administration reduces radiographic erosive progression in the subject. In some embodiments, the administration reduces joint space narrowing in the subject. In some embodiments, the administration reduces cartilage degradation in the subject. In some embodiments, the administration reduces the number of new erosive joints in the subject. In some embodiments, the administration reduces the number of new erosive joints in the subject at 48 weeks. In some embodiments, the administration inhibits the development of new erosive joints in the subject. In some embodiments, the administration reduces the number of ‘S/J’ IP joints that develop ‘E’ phases in the subject. In some embodiments, the administration reduces the number of ‘S/J’ IP joints that develop ‘E’ phases in the subject at 48 weeks. In some embodiments, the administration inhibits the development of ‘E’ phases in ‘S/J’ IP joints in the subject.

In some embodiments, the administration improves erosive IP finger joint remodeling in the subject. In some embodiments, the administration improves erosive IP finger joint remodeling in the subject at one year. In some embodiments, the administration improves erosive IP finger joint remodeling in the subject at two years. In some embodiments, the administration increases total GUSS™ in the subject. In some embodiments, the administration increases total GUSS™ in the subject at 12 weeks. In some embodiments, the administration increases total GUSS™ in the subject at 48 weeks.

In some embodiments, the administration reduces pain in the subject. In some embodiments, the administration reduces pain in the subject at 96 weeks. In some embodiments, pain reduction is assessed by numeric rating scale (NRS) pain.

In some embodiments, the administration improves joint function in the subject. In some embodiments, the administration improves joint function in the subject as assessed by the Australian-Canadian Hand Osteoarthritis Index (AUSCAN). In some embodiments, the administration improves joint function in the subject as assessed by the Functional Index for Hand Osteoarthritis (FIHOA).

In some embodiments, the subject does not exhibit a dose limiting toxicity (DLT) during denosumab administration. In some embodiments, the subject does not exhibit any grade 3 or grade 4 adverse events associated with denosumab during denosumab administration. In some embodiments, the subject is not hypocalcemic during denosumab administration.

In some embodiments, the subject exhibits at least one sign of clinical or sonographic inflammation prior to denosumab administration. In some embodiments, the subject exhibits at least one sign of clinical or sonographic inflammation in at least one interphalangeal finger joint prior to denosumab administration. In some embodiments, the subject exhibits at least one interphalangeal finger joint in the J or E phase with at least one inflammatory sign prior to denosumab administration. In some embodiments, the subject exhibits at least one interphalangeal finger joint in the J or E phase with palpable swelling prior to denosumab administration.

In some embodiments, the subject is at least about 18 years old. In some embodiments, the subject is at least about 30 years old. In some embodiments, the subject has not been diagnosed with vitamin D deficiency. In some embodiments, the subject has not been diagnosed with a chronic inflammatory rheumatic disease. In some embodiments, the subject has not been diagnosed with psoriasis. In some embodiments, the subject has not been diagnosed with cancer. In some embodiments, the subject has not been diagnosed with a chronic infectious disease.

In some embodiments, the subject has not been previously treated for erosive hand osteoarthritis. In some embodiments, the subject was previously treated for erosive hand osteoarthritis.

In some embodiments, the subject was previously treated with at least one therapeutic agent chosen from tumor necrosis factor α (TNFα) blocking agents, anti-interleukin-1a inhibitors, anti-interleukin-1β inhibitors, and combinations thereof. In some embodiments, the subject was previously treated with at least one therapeutic agent chosen from adalimumab, etanercept, lutikizumab, and combinations thereof.

Also disclosed herein is a pharmaceutical composition comprising denosumab for use in a method of treating erosive hand osteoarthritis in a subject in need thereof. In some embodiments, the method comprises administering to the subject denosumab in an amount equivalent to about 45 mg to about 60 mg (e.g., about 45 mg to less than 60 mg; about 45 mg to about 55 mg; about 45 mg to about 50 mg; about 45 mg; about 50 mg; about 55 mg; about 60 mg) denosumab administered once every about 3 months. In some embodiments, the method comprises administering to the subject denosumab in an amount equivalent to about 45 mg denosumab administered once every about 3 months. In some embodiments, the method comprises administering to the subject denosumab in an amount equivalent to about 50 mg denosumab administered once every about 3 months. In some embodiments, the method comprises administering to the subject denosumab in an amount equivalent to about 55 mg denosumab administered once every about 3 months. In some embodiments, the method comprises administering to the subject denosumab in an amount equivalent to about 60 mg denosumab administered once every about 3 months.

In some embodiments, the method comprises administering to the subject about 45 mg denosumab once every about 3 months. In some embodiments, the method comprises administering to the subject about 50 mg denosumab once every about 3 months. In some embodiments, the method comprises administering to the subject about 55 mg denosumab once every about 3 months. In some embodiments, the method comprises administering to the subject about 60 mg denosumab once every about 3 months.

In some embodiments, the amount of denosumab provides:
a. a mean scrum concentration of denosumab substantially similar to that achieved by administering 60 mg denosumab once every about 3 months; and/or
b. a mean plasma area under the curve (AUC_{0-∞}) substantially similar to that achieved by administering 60 mg denosumab once every about 3 months; and/or
c. a difference between the mean plasma Cₘₐₓ at steady state and a mean plasma Cₘᵢₙ at steady state substantially similar to that achieved by administering 60 mg denosumab once every about 3 months; and/or
d. a mean plasma Cₘₐₓ at steady state substantially similar to that achieved by administering denosumab 60 mg once every about 3 months.

In some embodiments, the amount of denosumab provides:
a. a mean serum concentration of denosumab substantially similar to that achieved by administering 55 mg denosumab once every about 3 months; and/or
b. a mean plasma area under the curve (AUC_{0-∞}) substantially similar to that achieved by administering 55 mg denosumab once every about 3 months; and/or
c. a difference between the mean plasma Cₘₐₓ at steady state and a mean plasma Cₘᵢₙ at steady state substantially similar to that achieved by administering 55 mg denosumab once every about 3 months; and/or
d. a mean plasma Cₘₐₓ at steady state substantially similar to that achieved by administering denosumab 55 mg once every about 3 months.

In some embodiments, the amount of denosumab provides:
a. a mean serum concentration of denosumab substantially similar to that achieved by administering 50 mg denosumab once every about 3 months; and/or
b. a mean plasma area under the curve (AUC_{0-∞}) substantially similar to that achieved by administering 50 mg denosumab once every about 3 months; and/or
c. a difference between the mean plasma Cₘₐₓ at steady state and a mean plasma Cₘᵢₙ at steady state substantially similar to that achieved by administering 50 mg denosumab once every about 3 months; and/or
d. a mean plasma Cₘₐₓ at steady state substantially similar to that achieved by administering denosumab 50 mg once every about 3 months.

In some embodiments, the amount of denosumab provides:
a. a mean scrum concentration of denosumab substantially similar to that achieved by administering 45 mg denosumab once every about 3 months; and/or
b. a mean plasma area under the curve (AUC_{0-∞}) substantially similar to that achieved by administering 45 mg denosumab once every about 3 months; and/or
c. a difference between the mean plasma Cₘₐₓ at steady state and a mean plasma Cₘᵢₙ at steady state substantially similar to that achieved by administering 45 mg denosumab once every about 3 months; and/or
d. a mean plasma Cₘₐₓ at steady state substantially similar to that achieved by administering denosumab 45 mg once every about 3 months.

In some embodiments, the subject is administered denosumab for at least 12 weeks. In some embodiments, the subject is administered denosumab for at least 24 weeks. In some embodiments, the subject is administered denosumab for at least 48 weeks. In some embodiments, the subject is administered denosumab for at least 96 weeks.

In some embodiments, the subject is administered denosumab once every about 3 months. In some embodiments, the subject is administered denosumab once every about 3 months for at least 2 cycles. In some embodiments, the subject is administered denosumab once every about 3 months for at least 3 cycles. In some embodiments, the subject is administered denosumab once every about 3 months for at least 4 cycles. In some embodiments, the subject is administered denosumab once every about 3 months for at least 5 cycles. In some embodiments, the subject is administered denosumab once every about 3 months for at least 6 cycles. In some embodiments, the subject is administered denosumab once every about 3 months for at least 7 cycles. In some embodiments, the subject is administered denosumab once every about 3 months for at least 8 cycles.

In some embodiments, denosumab is administered to the subject by injection. In some embodiments, denosumab is administered to the subject by subcutaneous injection. In some embodiments, denosumab is administered to the subject by injection into the upper arm, upper thigh, or abdomen of the subject. In some embodiments, denosumab is administered to the subject by subcutaneous injection into the upper arm, upper thigh, or abdomen of the subject.

In some embodiments, the method further comprises administering to the subject at least one therapeutic agent chosen from analgesics and non-steroidal anti-inflammatory drugs. In some embodiments, the method further comprises administering to the subject a stable dose of at least one therapeutic agent chosen from analgesics and non-steroidal anti-inflammatory drugs. In some embodiments, the method further comprises administering to the subject daily supplements of oral calcium and vitamin D₃. In some embodiments, the method further comprises administering to the subject daily supplements of oral calcium (1000 mg elemental calcium) and vitamin D₃ (880 international units).

In some embodiments, the administration reduces radiographic erosive progression in the subject. In some embodiments, the administration reduces joint space narrowing in the subject. In some embodiments, the administration reduces cartilage degradation in the subject. In some embodiments, the administration reduces the number of new erosive joints in the subject. In some embodiments, the administration reduces the number of new erosive joints in the subject at 48 weeks. In some embodiments, the administration inhibits the development of new erosive joints in the subject. In some embodiments, the administration reduces the number of ‘S/J’ IP joints that develop ‘E’ phases in the subject. In some embodiments, the administration reduces the number of ‘S/J’ IP joints that develop ‘E’ phases in the subject at 48 weeks. In some embodiments, the administration inhibits the development of ‘E’ phases in ‘S/J’ IP joints in the subject.

In some embodiments, the administration improves erosive IP finger joint remodeling in the subject. In some embodiments, the administration improves erosive IP finger joint remodeling in the subject at one year. In some embodiments, the administration improves erosive IP finger joint remodeling in the subject at two years. In some embodiments, the administration increases total GUSS™ in the subject. In some embodiments, the administration increases total GUSS™ in the subject at 12 weeks. In some embodiments, the administration increases total GUSS™ in the subject at 48 weeks.

In some embodiments, the administration reduces pain in the subject. In some embodiments, the administration reduces pain in the subject at 96 weeks. In some embodiments, pain reduction is assessed by numeric rating scale (NRS) pain.

In some embodiments, the administration improves joint function in the subject. In some embodiments, the administration improves joint function in the subject as assessed by the Australian-Canadian Hand Osteoarthritis Index (AUSCAN). In some embodiments, the administration improves joint function in the subject as assessed by the Functional Index for Hand Osteoarthritis (FIHOA).

In some embodiments, the subject does not exhibit a dose limiting toxicity (DLT) during denosumab administration. In some embodiments, the subject does not exhibit any grade 3 or grade 4 adverse events associated with denosumab during denosumab administration. In some embodiments, the subject is not hypocalcemic during denosumab administration.

In some embodiments, the subject exhibits at least one sign of clinical or sonographic inflammation prior to denosumab administration. In some embodiments, the subject exhibits at least one sign of clinical or sonographic inflammation in at least one interphalangeal finger joint prior to denosumab administration. In some embodiments, the subject exhibits at least one interphalangeal finger joint in the J or E phase with at least one inflammatory sign prior to denosumab administration. In some embodiments, the subject exhibits at least one interphalangeal finger joint in the J or E phase with palpable swelling prior to denosumab administration.

In some embodiments, the subject is at least about 18 years old. In some embodiments, the subject is at least about 30 years old. In some embodiments, the subject has not been diagnosed with vitamin D deficiency. In some embodiments, the subject has not been diagnosed with a chronic inflammatory rheumatic disease. In some embodiments, the subject has not been diagnosed with psoriasis. In some embodiments, the subject has not been diagnosed with cancer. In some embodiments, the subject has not been diagnosed with a chronic infectious disease.

In some embodiments, the subject has not been previously treated for erosive hand osteoarthritis. In some embodiments, the subject was previously treated for erosive hand osteoarthritis.

In some embodiments, the subject was previously treated with at least one therapeutic agent chosen from tumor necrosis factor α (TNFα) blocking agents, anti-interleukin-1α inhibitors, anti-interleukin-1β inhibitors, and combinations thereof. In some embodiments, the subject was previously treated with at least one therapeutic agent chosen from adalimumab, etanercept, lutikizumab, and combinations thereof.

Also disclosed herein is a method of reducing radiographic erosive progression, joint space narrowing, cartilage degradation, and/or bone formation in a subject in need thereof. In some embodiments, the method reduces radiographic erosive progression in the subject. In some embodiments, the method reduces joint space narrowing in the subject. In some embodiments, the method reduces cartilage degradation in the subject. In some embodiments, the method reduces bone formation in the subject. In some embodiments, the method possesses one or more of the features of a method described above.

Also disclosed herein is use of denosumab in the manufacture of a medicament adapted for use in a method of reducing radiographic erosive progression, joint space narrowing, cartilage degradation, and/or bone formation in a subject in need thereof. In some embodiments, the method reduces radiographic erosive progression in the subject. In some embodiments, the method reduces joint space narrowing in the subject. In some embodiments, the method reduces cartilage degradation in the subject. In some embodiments, the method reduces bone formation in the subject. In some embodiments, the method possesses one or more of the features of a method described above.

Also disclosed herein is a pharmaceutical composition comprising denosumab for use in a method of reducing radiographic erosive progression, joint space narrowing, cartilage degradation, and/or bone formation in a subject in need thereof. In some embodiments, the method reduces radiographic erosive progression in the subject. In some embodiments, the method reduces joint space narrowing in the subject. In some embodiments, the method reduces cartilage degradation in the subject. In some embodiments, the method reduces bone formation in the subject. In some embodiments, the method possesses one or more of the features of a method described above.

Also disclosed herein is a method of inhibiting the development of new erosive joints in a subject in need thereof. In some embodiments, the method inhibits the development of new erosive IP joints in the subject. In some embodiments, the method possesses one or more of the features of a method described above.

Also disclosed herein is use of denosumab in the manufacture of a medicament adapted for use in a method of inhibiting the development of new erosive joints in a subject in need thereof. In some embodiments, the method inhibits the development of new erosive IP joints in the subject. In some embodiments, the method possesses one or more of the features of a method described above.

Also disclosed herein is a pharmaceutical composition comprising denosumab for use in a method of inhibiting the development of new erosive joints in a subject in need thereof. In some embodiments, the method inhibits the development of new erosive IP joints in the subject. In some embodiments, the method possesses one or more of the features of a method described above.

Also disclosed herein is a method of reducing pain in a subject with erosive hand OA. In some embodiments, pain reduction in the subject is assessed by numeric rating scale (NRS) pain. In some embodiments, the method possesses one or more of the features of a method described above.

Also disclosed herein is use of denosumab in the manufacture of a medicament adapted for use in a method of reducing pain in a subject with erosive hand OA. In some embodiments, the method possesses one or more of the features of a method described above.

Also disclosed herein is a pharmaceutical composition comprising denosumab for use in a method of reducing pain in a subject with erosive hand OA. In some embodiments, the method possesses one or more of the features of a method described above.

### BRIEF DESCRIPTION OF THE DRAWINGS

FIG. 1A is a schematic detailing dosing regimens and assessments for a clinical trial described in Example 1. Patients were randomly assigned, in a 1:1 ratio, to receive subcutaneous injections of 60 mg of denosumab or placebo every 12 weeks for 48 weeks during the placebo-controlled double-blind phase of the study. Patients then received open-label denosumab administered subcutaneously at a dose of 60 mg every 12 weeks for an additional 48 weeks. The initial group assignment was still blinded. After the 96-week trial period, patients continued to receive open-label denosumab in a 1-year extension phase (data not shown).

FIG. 1B is an illustration of the trial profile.

FIGS. 2A and 2B are bar charts depicting the mean change GUSS™ (FIG. 2A) and percentage of new erosive joints (FIB. 2B) at weeks 24, 48, 72 and 96. The primary endpoint was the mean total GUSS™ at week 24 (FIG. 2A). Analysis performed in the intention-to-treat population: comparison between groups was done by Generalized Estimation Equations at joint level, accounting for within-patient clustering and adjusted for baseline unbalances. Missing values were imputed according to a predefined imputation model, including randomization group, baseline value and values at other time points available, presence of baseline inflammation, baseline number of affected joints. A indicates estimated mean difference from baseline. * indicates the estimated difference from week 48. The secondary endpoint was the cumulative incidence of new erosive joints at weeks 24 and 48 (FIG. 2B). New erosive joints were defined as joints that entered E or E/R phase by Verbruggen and Veys (19) but were in N, S of J phase at baseline. Odds ratio for erosive progression between groups and P values are shown.

FIGS. 3A and 3B are bar charts depicting changes in clinical data through the placebo-controlled and open-label extension phase. FIG. 3A shows the mean numeric rating scale (NRS) pain, ranging from 0 to 10, with higher scores indicating more pain. FIG. 3B shows the mean Functional Index for Hand Osteoarthritis (FIHOA), ranging from 0 to 30, where higher scores indicate more disability. All the data are shown for the full analysis set, which included all the patients who underwent randomization and received at least one dose of denosumab or placebo. P-values represent the comparison with placebo adjusted for baseline values by Generalized Estimation Equations.

FIG. 4 is a graph showing mean percentage changes from baseline in serum levels of C-telopeptide over time according to treatment groups (open square: placebo; closed circle: 6 mg of denosumab; closed triangle: 14 mg of denosumab; closed diamond: 30 mg of denosumab, open circle: 70 mg of alendronate weekly). The three-day time points are not drawn to scale on the x axis. Bars denote standard errors.

### DETAILED DESCRIPTION

Some embodiments of the present disclosure relate to methods of treating erosive hand osteoarthritis (OA) in a subject in need thereof, methods of reducing radiographic erosive progression, joint space narrowing, cartilage degradation, and/or bone formation in a subject in need thereof (e.g., a subject suffering from erosive hand OA), methods of inhibiting the development of new erosive joints in a subject in need thereof (e.g., a subject suffering from erosive hand OA), and methods of reducing pain in a subject with erosive hand OA. Additional embodiments of this disclosure relate to uses of denosumab in the manufacture of a medicament adapted for use in a method described herein, as well as pharmaceutical compositions comprising denosumab for use in a method described herein.

### Definitions

As used herein, the terms “a,” “an,” “the,” and similar referents, in the context of example embodiments and claims, are to be construed as covering both the singular and the plural, unless otherwise indicated herein or clearly contradicted by context.

As used herein, the term “and/or,” in the context of selections such as “[A] and/or [B],” includes [A] alone, [B] alone, and both [A] and [B].

As used herein, the terms “comprising,” “having,” “including,” and “containing” are to be construed as open-ended terms including the indicated component(s) but not excluding other elements (i.e., meaning “including, but not limited to”) unless otherwise noted.

As used herein, the term “about,” when used in connection with a dose or amount, include the value of a specified dose or amount or a range encompassing the dose or amount that is recognized by one of ordinary skill in the art to provide an effect equivalent to that obtained from the specified dose or amount. In some embodiments, the term “about” reflects a variation of 10% of a stated value. In some embodiments, the term “about” reflects a variation of 5% of a stated value. In some embodiments, the term “about” reflects a variation of 2% of a stated value. In some embodiments, the term “about” reflects a variation of 1% of a stated value.

As used herein, the term “administer” and its cognates (e.g., “administering”) includes both self-administration and administration to the patient by another person (e.g., a medical professional or caretaker).

As used herein, the term “antibody” refers to a protein having a conventional immunoglobulin format, comprising heavy and light chains, and comprising variable and constant regions. As a non-limiting example, an antibody may be an IgG which is a “Y-shaped” structure of two identical pairs of polypeptide chains, each pair having one “light” (e.g., having a molecular weight of about 25 kDa) and one “heavy” chain (e.g., having a molecular weight of about 50-70 kDa). As used herein, an “antibody” has a variable region and a constant region. In IgG formats, the variable region is generally about 100-110 or more amino acids, comprises three complementarity determining regions (CDRs), is primarily responsible for antigen recognition, and substantially varies among other antibodies that bind to different antigens. The constant region allows the antibody to recruit cells and molecules of the immune system. The variable region is made of the N-terminal regions of each light chain and heavy chain, while the constant region is made of the C-terminal portions of each of the heavy and light chains. (Janeway et al., “Structure of the Antibody Molecule and the Immunoglobulin Genes”, Immunobiology: The Immune System in Health and Disease, 4th ed. Elsevier Science Ltd./Garland Publishing, (1999)).

The general structure and properties of CDRs of antibodies have been described in the art. Briefly, in an antibody scaffold, the CDRs are embedded within a framework in the heavy and light chain variable region where they constitute the regions largely responsible for antigen binding and recognition. A variable region typically comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342:877-883), within a framework region (designated framework regions 1-4, FR1, FR2, FR3, and FR4, by Kabat et al., 1991; see also Chothia and Lesk, 1987, supra).

Antibodies can comprise any constant region known in the art. Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the present disclosure include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in some embodiments, the antibody is an antibody of isotype IgA, IgD, IgE, IgG, or IgM, including any one of IgG1, IgG2, IgG3 or IgG4.

As used herein, the term “chimeric antibody” refers to an antibody containing domains from two or more different antibodies. A chimeric antibody can, for example, contain the constant domains from one species and the variable domains from a second, or more generally, can contain stretches of amino acid sequence from at least two species. A chimeric antibody also may contain domains of two or more different antibodies within the same species.

As used herein, the term “humanized,” when used in relation to an antibody, refers to an antibody having at least CDR regions from a non-human source which are engineered to have a structure and immunological function more similar to true human antibodies than the original source antibodies. As a non-limiting example, humanizing can involve grafting a CDR from a non-human antibody, such as, e.g., a mouse antibody, into a human antibody. Humanizing also can involve select amino acid substitutions to make a non-human sequence more similar to a human sequence.

As used herein, the term “conservative amino acid substitution” refers to the substitution of one amino acid with another amino acid having similar properties, e.g., size, charge, hydrophobicity, hydrophilicity, and/or aromaticity, and includes exchanges within one of the following five groups:
I. Small aliphatic, nonpolar or slightly polar residues: Ala, Ser, Thr, Pro, Gly;
II. Polar, negatively charged residues and their amides and esters: Asp, Asn, Glu, Gln, cysteic acid and homocysteic acid;
III. Polar, positively charged residues: His, Arg, Lys; Ornithine (Orn)
IV. Large, aliphatic, nonpolar residues: Met, Leu, Ile, Val, Cys, Norleucine (Nle), homocysteine
V. Large, aromatic residues: Phe, Tyr, Trp, acetyl phenylalanine.

As used herein, the terms “at [X] week(s)” and “at [Y] year(s)” refer to a specified time point. Illustratively, an administration that improves erosive IP finger joint remodeling in the subject at [Y] year(s) refers to an administration that results in improved erosive IP finger joint remodeling as measured after [Y] year(s) of treatment when compared to a baseline condition.

The terms “at least one” and “one or more” are used interchangeably herein and include one of an indicated component(s) and more than onc (e.g., two, three, four, etc.) of an indicated component(s).

As used herein, the term “denosumab in an amount equivalent to [dose X] denosumab administered [on schedule Y]” refers to a denosumab dosing regimen (i.e., a dose A of denosumab administered on a schedule B) that produces a pharmacokinetic profile in a subject that is substantially similar in one or more features (e.g., a mean serum concentration; a mean plasma area under the curve (AUC_{0-∞}); a difference between the mean plasma Cₘₐₓ at steady state and a mean plasma Cₘᵢₙ at steady state; a mean plasma Cₘₐₓ at steady state) to a pharmacokinetic profile in a subject administered [dose X] denosumab [on schedule Y].

The terms “denosumab administration” and “denosumab treatment,” when used in connection with a time period, refer to a time period in which a subject is administered denosumab according to a specific schedule (i.e., from the first dose to the end of the final cycle). As a non-limiting example, a “denosumab administration” or “denosumab treatment” time period is 6 months from the first dose for a subject administered denosumab once every about 3 months for two cycles.

As used herein, “erosive hand osteoarthritis” is defined as radiographic evidence of ≥1 IP joint in J (i.e., loss of joint space) or E (i.e., subchondral erosion(s)) phase according to the Verbruggen and Veys anatomical phase scoring system.

As used herein, the term “previously treated,” when used in connection with a subject, refers to a subject that was administered a specified therapy or therapies at least once prior to the first dose of RANKL inhibitor (e.g., denosumab) being administered to the subject.

As used herein, the terms “subject” and “patient” are used interchangeably and refer to an animal, e.g., a human. In some embodiments, the “subject” or “patient” is a human.

As used herein, the term “substantially similar,” used in connection with a specific property, includes the specific property and properties recognized by one of ordinary skill in the art as providing an equivalent effect to the specific property. In some embodiments, when used in connection with a quantitative property, the term “substantially similar” reflects a variation of 10% of the numerical value(s) corresponding to the quantitative property. In some embodiments, when used in connection with a quantitative property, the term “substantially similar” reflects a variation of 5% of the numerical value(s) corresponding to the quantitative property. In some embodiments, when used in connection with a quantitative property, the term “substantially similar” reflects a variation of 2% of the numerical value(s) corresponding to the quantitative property. In some embodiments, when used in connection with a quantitative property, the term “substantially similar” reflects a variation of 1% of numerical value(s) corresponding to the quantitative property.

As used herein, the term “treatment” and its cognates (e.g., “treat” or “treating”) refer to improving at least one sign or symptom of a disease in a subject, delaying the onset of at least one sign or symptom of a disease in a subject, or lessening the severity of at least one sign or symptom of a disease. “Treatment” and its cognates do not necessarily imply 100% or complete treatment. Rather, there are varying degrees of treatment that one of ordinary skill in the art would recognize as having a potential benefit or therapeutic effect. “Treatment” and its cognates, as used herein in the context of erosive hand OA, include, but are not limited, to the following: reduced radiographic erosive progression; reduced joint space narrowing; reduced cartilage degradation; reduced bone formation; inhibition of the development of new erosive joints; and pain reduction. Improvements in or lessening the severity of any of these signs or symptoms can be readily assessed according to methods and techniques known in the art or those subsequently developed.

### Non-Limiting Example Embodiments #1

Without limitation, some example embodiments of the present disclosure include:
1. A method of treating erosive hand osteoarthritis (OA) in a subject in need thereof, comprising administering to the subject denosumab in an amount equivalent to about 45 mg to about 60 mg (e.g., about 45 mg to less than 60 mg; about 45 mg to about 55 mg; about 45 mg to about 50 mg; about 45 mg; about 50 mg; about 55 mg; about 60 mg) denosumab administered once every about 3 months.
2. A method of reducing radiographic erosive progression, joint space narrowing, cartilage degradation, and/or bone formation in a subject in need thereof, comprising administering to the subject denosumab in an amount equivalent to about 45 mg to about 60 mg (e.g., about 45 mg to less than 60 mg; about 45 mg to about 55 mg; about 45 mg to about 50 mg; about 45 mg; about 50 mg; about 55 mg; about 60 mg) denosumab administered once every about 3 months.
3. The method according to Embodiment 2, wherein the subject has been diagnosed with erosive hand osteoarthritis (OA).
4. The method according to any one of Embodiments 1 to 3, wherein the administration reduces radiographic erosive progression in the subject.
5. The method according to any one of Embodiments 1 to 4, wherein the administration reduces joint space narrowing in the subject.
6. The method according to any one of Embodiments 1 to 5, wherein the administration reduces cartilage degradation in the subject.
7. The method according to any one of Embodiments 1 to 6, wherein the administration reduces bone formation in the subject.
8. A method of inhibiting the development of new erosive joints in a subject in need thereof, comprising administering to the subject denosumab in an amount equivalent to about 45 mg to about 60 mg (e.g., about 45 mg to less than 60 mg; about 45 mg to about 55 mg; about 45 mg to about 50 mg; about 45 mg; about 50 mg; about 55 mg; about 60 mg) denosumab administered once every about 3 months.
9. The method according to Embodiment 8, wherein the subject has been diagnosed with erosive hand osteoarthritis (OA).
10. The method according to any one of Embodiments 1 to 9, wherein the administration inhibits the development of new erosive IP joints in the subject (e.g., at 48 weeks).
11. The method according to any one of Embodiments 1 to 10, wherein the administration reduces the number of ‘S/J’ IP joints that develop ‘E’ phases in the subject.
12. The method according to any one of Embodiments 1 to 11, wherein the administration inhibits the development of ‘E’ phases in ‘S/J’ IP joints in the subject.
13. A method of reducing pain in a subject with erosive hand osteoarthritis (OA), comprising administering to the subject denosumab in an amount equivalent to about 45 mg to about 60 mg (e.g., about 45 mg to less than 60 mg; about 45 mg to about 55 mg; about 45 mg to about 50 mg; about 45 mg; about 50 mg; about 55 mg; about 60 mg) denosumab administered once every about 3 months.
14. The method according to any one of Embodiments 1 to 13, wherein the administration reduces pain in the subject as assessed by numeric rating scale (NRS) pain.
15. The method according to any one of Embodiments 1 to 14, wherein the administration improves erosive IP finger joint remodeling in the subject (e.g., at one year; at two years).
16. The method according to any one of Embodiments 1 to 15, wherein the administration increases total GUSS™ in the subject (e.g., at 12 weeks; at 48 weeks).
17. The method according to any one of Embodiments 1 to 16, wherein the administration improves joint function in the subject.
18. The method according to any one of Embodiments 1 to 17, wherein the administration improves joint function in the subject as assessed by the Australian-Canadian Hand Osteoarthritis Index (AUSCAN).
19. The method according to any one of Embodiments 1 to 18, wherein the administration improves joint function in the subject as assessed by the Functional Index for Hand Osteoarthritis (FIHOA).
20. The method according to any one of Embodiments 1 to 19, wherein the subject does not exhibit a dose limiting toxicity (DLT) during denosumab administration.
21. The method according to any one of Embodiments 1 to 20, wherein the subject does not exhibit any grade 3 or grade 4 adverse events associated with denosumab during denosumab administration.
22. The method according to any one of Embodiments 1 to 21, wherein the subject is not hypocalcemic immediately prior to or during denosumab administration.
23. The method according to any one of Embodiments 1 to 22, comprising administering to the subject denosumab in an amount equivalent to about 45 mg to less than 60 mg denosumab administered once every about 3 months.
24. The method according to any one of Embodiments 1 to 22, comprising administering to the subject denosumab in an amount equivalent to about 45 mg denosumab administered once every about 3 months.
25. The method according to any one of Embodiments 1 to 22, comprising administering to the subject denosumab in an amount equivalent to about 50 mg denosumab administered once every about 3 months.
26. The method according to any one of Embodiments 1 to 22, comprising administering to the subject denosumab in an amount equivalent to about 55 mg denosumab administered once every about 3 months.
27. The method according to any one of Embodiments 1 to 22, comprising administering to the subject denosumab in an amount equivalent to about 60 mg denosumab administered once every about 3 months.
28. The method according to any one of Embodiments 1 to 22, comprising administering to the subject about 45 mg denosumab once every about 3 months.
29. The method according to any one of Embodiments 1 to 22, comprising administering to the subject about 50 mg denosumab once every about 3 months.
30. The method according to any one of Embodiments 1 to 22, comprising administering to the subject about 55 mg denosumab once every about 3 months.
31. The method according to any one of Embodiments 1 to 22, comprising administering to the subject about 60 mg denosumab once every about 3 months.
32. The method according to any one of Embodiments 1 to 22, wherein the amount of denosumab provides:
   a. a mean serum concentration of denosumab substantially similar to that achieved by administering 60 mg denosumab once every about 3 months; and/or
   b. a mean plasma area under the curve (AUC_{0-∞}) substantially similar to that achieved by administering 60 mg denosumab once every about 3 months; and/or
   c. a difference between the mean plasma Cₘₐₓ at steady state and a mean plasma Cₘᵢₙ at steady state substantially similar to that achieved by administering 60 mg denosumab once every about 3 months; and/or
   d. a mean plasma Cₘₐₓ at steady state substantially similar to that achieved by administering denosumab 60 mg once every about 3 months.
33. The method according to any one of Embodiments 1 to 22, wherein the amount of denosumab provides:
   a. a mean serum concentration of denosumab substantially similar to that achieved by administering 55 mg denosumab once every about 3 months; and/or
   b. a mean plasma area under the curve (AUC_{0-∞}) substantially similar to that achieved by administering 55 mg denosumab once every about 3 months; and/or
   c. a difference between the mean plasma Cₘₐₓ at steady state and a mean plasma Cₘᵢₙ at steady state substantially similar to that achieved by administering 55 mg denosumab once every about 3 months; and/or
   d. a mean plasma Cₘₐₓ at steady state substantially similar to that achieved by administering denosumab 55 mg once every about 3 months.
34. The method according to any one of Embodiments 1 to 22, wherein the amount of denosumab provides:
   a. a mean serum concentration of denosumab substantially similar to that achieved by administering 50 mg denosumab once every about 3 months; and/or
   b. a mean plasma area under the curve (AUC_{0-∞}) substantially similar to that achieved by administering 50 mg denosumab once every about 3 months; and/or
   c. a difference between the mean plasma Cₘₐₓ at steady state and a mean plasma Cₘᵢₙ at steady state substantially similar to that achieved by administering 50 mg denosumab once every about 3 months; and/or
   d. a mean plasma Cₘₐₓ at steady state substantially similar to that achieved by administering denosumab 50 mg once every about 3 months.
35. The method according to any one of Embodiments 1 to 22, wherein the amount of denosumab provides:
   a. a mean serum concentration of denosumab substantially similar to that achieved by administering 45 mg denosumab once every about 3 months; and/or
   b. a mean plasma area under the curve (AUC_{0-∞}) substantially similar to that achieved by administering 45 mg denosumab once every about 3 months; and/or
   c. a difference between the mean plasma Cₘₐₓ at steady state and a mean plasma Cₘᵢₙ at steady state substantially similar to that achieved by administering 45 mg denosumab once every about 3 months; and/or
   d. a mean plasma Cₘₐₓ at steady state substantially similar to that achieved by administering denosumab 45 mg once every about 3 months.
36. The method according to any one of Embodiments 1 to 35, wherein the subject is administered denosumab for at least 12 weeks.
37. The method according to any one of Embodiments 1 to 36, wherein the subject is administered denosumab for at least 24 weeks.
38. The method according to any one of Embodiments 1 to 37, wherein the subject is administered denosumab for at least 48 weeks.
39. The method according to any one of Embodiments 1 to 38, wherein the subject is administered denosumab for at least 96 weeks.
40. The method according to any one of Embodiments 1 to 39, wherein the subject is administered denosumab once every about 3 months.
41. The method according to any one of Embodiments 1 to 40, wherein the subject is administered denosumab once every about 3 months for at least 2 cycles (e.g., 2 cycles; 3 cycles; 4 cycles; 5 cycles; 6 cycles; 7 cycles; 8 cycles; at least 2 cycles; at least 3 cycles; at least 4 cycles; at least 5 cycles; at least 6 cycles; at least 7 cycles; at least 8 cycles).
42. The method according to any one of Embodiments 1 to 41, wherein denosumab is administered to the subject by injection (e.g., by subcutaneous injection).
43. The method according to any one of Embodiments 1 to 42, wherein denosumab is administered to the subject by injection (e.g., by subcutaneous injection) into the upper arm, upper thigh, or abdomen of the subject.
44. The method according to any one of Embodiments 1 to 43, wherein the method further comprises administering to the subject at least one therapeutic agent chosen from analgesics and non-steroidal anti-inflammatory drugs.
45. The method according to any one of Embodiments 1 to 44, wherein the method further comprises administering to the subject a stable dose of at least one therapeutic agent chosen from analgesics and non-steroidal anti-inflammatory drugs.
46. The method according to any one of Embodiments 1 to 45, wherein the method further comprises administering to the subject daily supplements of oral calcium and vitamin D3 (e.g., daily supplements of oral calcium (1000 mg elemental calcium) and vitamin D₃ (880 international units)).
47. The method according to any one of Embodiments 1 to 46, wherein the subject exhibits at least one sign of clinical or sonographic inflammation (e.g., in at least one interphalangeal finger joint) prior to denosumab administration.
48. The method according to any one of Embodiments 1 to 47, wherein the subject exhibits at least one interphalangeal finger joint in the J or E phase with at least one inflammatory sign prior to denosumab administration.
49. The method according to any one of Embodiments 1 to 48, wherein the subject exhibits at least one interphalangeal finger joint in the J or E phase with palpable swelling prior to denosumab administration.
50. The method according to any one of Embodiments 1 to 49, wherein the subject is at least about 18 years old (e.g., at least about 30 years old).
51. The method according to any one of Embodiments 1 to 50, wherein the subject has not been diagnosed with vitamin D deficiency, a chronic inflammatory rheumatic disease, psoriasis, cancer, or a chronic infectious disease.
52. The method according to any one of Embodiments 1 to 51, wherein the subject has not been previously treated for erosive hand osteoarthritis.
53. The method according to any one of Embodiments 1 to 51, wherein the subject was previously treated for erosive hand osteoarthritis.
54. The method according to Embodiment 53, wherein the subject was previously treated with at least one therapeutic agent chosen from tumor necrosis factor α (TNFα) blocking agents, anti-interleukin-1a inhibitors, anti-interleukin-1β inhibitors, and combinations thereof.
55. The method according to Embodiment 53 or 54, wherein the subject was previously treated with at least one therapeutic agent chosen from adalimumab, etanercept, lutikizumab, and combinations thereof.

### Non-Limiting Example Embodiments #2

Without limitation, some example embodiments/features of the present disclosure include:
1. Use of denosumab in the manufacture of a medicament adapted for use in a method of treating erosive hand osteoarthritis (OA) in a subject in need thereof, wherein the method comprises administering to the subject denosumab in an amount equivalent to about 45 mg to about 60 mg (e.g., about 45 mg to less than 60 mg; about 45 mg to about 55 mg; about 45 mg to about 50 mg; about 45 mg; about 50 mg; about 55 mg; about 60 mg) denosumab administered once every about 3 months.
2. Use of denosumab in the manufacture of a medicament adapted for use in a method of reducing radiographic erosive progression, joint space narrowing, cartilage degradation, and/or bone formation in a subject in need thereof, wherein the method comprises administering to the subject denosumab in an amount equivalent to about 45 mg to about 60 mg (e.g., about 45 mg to less than 60 mg; about 45 mg to about 55 mg; about 45 mg to about 50 mg; about 45 mg; about 50 mg; about 55 mg; about 60 mg) denosumab administered once every about 3 months.
3. The use according to Feature 2, wherein the subject has been diagnosed with erosive hand osteoarthritis (OA).
4. The use according to any one of Features 1 to 3, wherein the administration reduces radiographic erosive progression in the subject.
5. The use according to any one of Features 1 to 4, wherein the administration reduces joint space narrowing in the subject.
6. The use according to any one of Features 1 to 5, wherein the administration reduces cartilage degradation in the subject.
7. The use according to any one of Features 1 to 6, wherein the administration reduces bone formation in the subject.
8. Use of denosumab in the manufacture of a medicament adapted for use in a method of inhibiting the development of new erosive joints in a subject in need thereof, wherein the method comprises administering to the subject denosumab in an amount equivalent to about 45 mg to about 60 mg (e.g., about 45 mg to less than 60 mg; about 45 mg to about 55 mg; about 45 mg to about 50 mg; about 45 mg; about 50 mg; about 55 mg; about 60 mg) denosumab administered once every about 3 months.
9. The use according to Feature 8, wherein the subject has been diagnosed with erosive hand osteoarthritis (OA).
10. The use according to any one of Features 1 to 9, wherein the administration inhibits the development of new erosive IP joints in the subject (e.g., at 48 weeks).
11. The use according to any one of Features 1 to 10, wherein the administration reduces the number of ‘S/J’ IP joints that develop ‘E’ phases in the subject.
12. The use according to any one of Features 1 to 11, wherein the administration inhibits the development of ‘E’ phases in ‘S/J’ IP joints in the subject.
13. Use of denosumab in the manufacture of a medicament adapted for use in a method of reducing pain in a subject with erosive hand osteoarthritis (OA), wherein the method comprises administering to the subject denosumab in an amount equivalent to about 45 mg to about 60 mg (e.g., about 45 mg to less than 60 mg; about 45 mg to about 55 mg; about 45 mg to about 50 mg; about 45 mg; about 50 mg; about 55 mg; about 60 mg) denosumab administered once every about 3 months.
14. The use according to any one of Features 1 to 13, wherein the administration reduces pain in the subject as assessed by numeric rating scale (NRS) pain.
15. The use according to any one of Features 1 to 14, wherein the administration improves erosive IP finger joint remodeling in the subject (e.g., at one year; at two years).
16. The use according to any one of Features 1 to 15, wherein the administration increases total GUSS™ in the subject (e.g., at 12 weeks; at 48 weeks).
17. The use according to any one of Features 1 to 16, wherein the administration improves joint function in the subject.
18. The use according to any one of Features 1 to 17, wherein the administration improves joint function in the subject as assessed by the Australian-Canadian Hand Osteoarthritis Index (AUSCAN).
19. The use according to any one of Features 1 to 18, wherein the administration improves joint function in the subject as assessed by the Functional Index for Hand Osteoarthritis (FIHOA).
20. The use according to any one of Features 1 to 19, wherein the subject does not exhibit a dose limiting toxicity (DLT) during denosumab administration.
21 The use according to any one of Features 1 to 20, wherein the subject does not exhibit any grade 3 or grade 4 adverse events associated with denosumab during denosumab administration.
22. The use according to any one of Features 1 to 21, wherein the subject is not hypocalcemic immediately prior to or during denosumab administration.
23. The use according to any one of Features 1 to 22, wherein the method comprises administering to the subject denosumab in an amount equivalent to about 45 mg to less than 60 mg denosumab administered once every about 3 months.
24. The use according to any one of Features 1 to 22, wherein the method comprises administering to the subject denosumab in an amount equivalent to about 45 mg denosumab administered once every about 3 months.
25. The use according to any one of Features 1 to 22, wherein the method comprises administering to the subject denosumab in an amount equivalent to about 50 mg denosumab administered once every about 3 months.
26. The use according to any one of Features 1 to 22, wherein the method comprises administering to the subject denosumab in an amount equivalent to about 55 mg denosumab administered once every about 3 months.
27 The use according to any one of Features 1 to 22, wherein the method comprises administering to the subject denosumab in an amount equivalent to about 60 mg denosumab administered once every about 3 months.
28. The use according to any one of Features 1 to 22, wherein the method comprises administering to the subject about 45 mg denosumab once every about 3 months.
29. The use according to any one of Features 1 to 22, wherein the method comprises administering to the subject about 50 mg denosumab once every about 3 months.
30. The use according to any one of Features 1 to 22, wherein the method comprises administering to the subject about 55 mg denosumab once every about 3 months.
31. The use according to any one of Features 1 to 22, wherein the method comprises administering to the subject about 60 mg denosumab once every about 3 months.
32. The use according to any one of Features 1 to 22, wherein the amount of denosumab provides:
   a. a mean serum concentration of denosumab substantially similar to that achieved by administering 60 mg denosumab once every about 3 months; and/or
   b. a mean plasma area under the curve (AUC_{0-∞}) substantially similar to that achieved by administering 60 mg denosumab once every about 3 months; and/or
   c. a difference between the mean plasma Cₘₐₓ at steady state and a mean plasma Cₘᵢₙ at steady state substantially similar to that achieved by administering 60 mg denosumab once every about 3 months; and/or
   d. a mean plasma Cₘₐₓ at steady state substantially similar to that achieved by administering denosumab 60 mg once every about 3 months.
33. The use according to any one of Features 1 to 22, wherein the amount of denosumab provides:
   a. a mean serum concentration of denosumab substantially similar to that achieved by administering 55 mg denosumab once every about 3 months; and/or
   b. a mean plasma area under the curve (AUC_{0-∞}) substantially similar to that achieved by administering 55 mg denosumab once every about 3 months; and/or
   c. a difference between the mean plasma Cₘₐₓ at steady state and a mean plasma Cₘᵢₙ at steady state substantially similar to that achieved by administering 55 mg denosumab once every about 3 months; and/or
   d. a mean plasma Cₘₐₓ at steady state substantially similar to that achieved by administering denosumab 55 mg once every about 3 months.
34. The use according to any one of Features 1 to 22, wherein the amount of denosumab provides:
   a. a mean serum concentration of denosumab substantially similar to that achieved by administering 50 mg denosumab once every about 3 months; and/or
   b. a mean plasma area under the curve (AUC_{0-∞}) substantially similar to that achieved by administering 50 mg denosumab once every about 3 months; and/or
   c. a difference between the mean plasma Cₘₐₓ at steady state and a mean plasma Cₘᵢₙ at steady state substantially similar to that achieved by administering 50 mg denosumab once every about 3 months; and/or
   d. a mean plasma Cₘₐₓ at steady state substantially similar to that achieved by administering denosumab 50 mg once every about 3 months.
35. The use according to any one of Features 1 to 22, wherein the amount of denosumab provides:
   a. a mean serum concentration of denosumab substantially similar to that achieved by administering 45 mg denosumab once every about 3 months; and/or
   b. a mean plasma area under the curve (AUC_{0-∞}) substantially similar to that achieved by administering 45 mg denosumab once every about 3 months; and/or
   c. a difference between the mean plasma Cₘₐₓ at steady state and a mean plasma Cₘᵢₙ at steady state substantially similar to that achieved by administering 45 mg denosumab once every about 3 months; and/or
   d. a mean plasma Cₘₐₓ at steady state substantially similar to that achieved by administering denosumab 45 mg once every about 3 months.
36. The use according to any one of Features 1 to 35, wherein the subject is administered denosumab for at least 12 weeks.
37. The use according to any one of Features 1 to 36, wherein the subject is administered denosumab for at least 24 weeks.
38. The use according to any one of Features 1 to 37, wherein the subject is administered denosumab for at least 48 weeks.
39 The use according to any one of Features 1 to 38, wherein the subject is administered denosumab for at least 96 weeks.
40. The use according to any one of Features 1 to 39, wherein the subject is administered denosumab once every about 3 months.
41. The use according to any one of Features 1 to 40, wherein the subject is administered denosumab once every about 3 months for at least 2 cycles (e.g., 2 cycles; 3 cycles; 4 cycles; 5 cycles; 6 cycles; 7 cycles; 8 cycles; at least 2 cycles; at least 3 cycles; at least 4 cycles; at least 5 cycles; at least 6 cycles; at least 7 cycles; at least 8 cycles).
42. The use according to any one of Features 1 to 41, wherein the medicament is adapted for administration to the subject by injection (e.g., by subcutaneous injection).
43. The use according to any one of Features 1 to 42, wherein the medicament is adapted for administration to the subject by injection (e.g., by subcutaneous injection) into the upper arm, upper thigh, or abdomen of the subject.
44. The use according to any one of Features 1 to 43, wherein the method further comprises administering to the subject at least one therapeutic agent chosen from analgesics and non-steroidal anti-inflammatory drugs.
45. The use according to any one of Features 1 to 44, wherein the method further comprises administering to the subject a stable dose of at least one therapeutic agent chosen from analgesics and non-steroidal anti-inflammatory drugs.
46. The use according to any one of Features 1 to 45, wherein the method further comprises administering to the subject daily supplements of oral calcium and vitamin D3 (e.g., daily supplements of oral calcium (1000 mg elemental calcium) and vitamin D₃ (880 international units)).
47. The use according to any one of Features 1 to 46, wherein the subject exhibits at least one sign of clinical or sonographic inflammation (e.g., in at least one interphalangeal finger joint) prior to denosumab administration.
48. The use according to any one of Features 1 to 47, wherein the subject exhibits at least one interphalangeal finger joint in the J or E phase with at least one inflammatory sign prior to denosumab administration.
49. The use according to any one of Features 1 to 48, wherein the subject exhibits at least one interphalangeal finger joint in the J or E phase with palpable swelling prior to denosumab administration.
50. The use according to any one of Features 1 to 49, wherein the subject is at least about 18 years old (e.g., at least about 30 years old).
51. The use according to any one of Features 1 to 50, wherein the subject has not been diagnosed with vitamin D deficiency, a chronic inflammatory rheumatic disease, psoriasis, cancer, or a chronic infectious disease.
52. The use according to any one of Features 1 to 51, wherein the subject has not been previously treated for erosive hand osteoarthritis.
53. The use according to any one of Features 1 to 51, wherein the subject was previously treated for erosive hand osteoarthritis.
54. The use according to Feature 53, wherein the subject was previously treated with at least one therapeutic agent chosen from tumor necrosis factor α (TNFα) blocking agents, anti-interleukin-la inhibitors, anti-interleukin-1β inhibitors, and combinations thereof.
55. The use according to Feature 53 or 54, wherein the subject was previously treated with at least one therapeutic agent chosen from adalimumab, etanercept, lutikizumab, and combinations thereof.

### Non-Limiting Example Embodiments #3

Without limitation, some example embodiments/clauses of the present disclosure include:

1. A pharmaceutical composition comprising denosumab for use in a method of treating erosive hand osteoarthritis (OA) in a subject in need thereof, wherein the method comprises administering to the subject denosumab in an amount equivalent to about 45 mg to about 60 mg (e.g., about 45 mg to less than 60 mg; about 45 mg to about 55 mg; about 45 mg to about 50 mg; about 45 mg; about 50 mg; about 55 mg; about 60 mg) denosumab administered once every about 3 months.
2. A pharmaceutical composition comprising denosumab for use in a method of reducing radiographic erosive progression, joint space narrowing, cartilage degradation, and/or bone formation in a subject in need thereof, wherein the method comprises administering to the subject denosumab in an amount equivalent to about 45 mg to about 60 mg (e.g., about 45 mg to less than 60 mg; about 45 mg to about 55 mg; about 45 mg to about 50 mg; about 45 mg; about 50 mg; about 55 mg; about 60 mg) denosumab administered once every about 3 months.
3. The pharmaceutical composition for use according to Clause 2, wherein the subject has been diagnosed with erosive hand osteoarthritis (OA).
4. The pharmaceutical composition for use according to any one of Clauses 1 to 3, wherein the administration reduces radiographic erosive progression in the subject.
5. The pharmaceutical composition for use according to any one of Clauses 1 to 4, wherein the administration reduces joint space narrowing in the subject.
6. The pharmaceutical composition for use according to any one of Clauses 1 to 5, wherein the administration reduces cartilage degradation in the subject.
7. The pharmaceutical composition for use according to any one of Clauses 1 to 6, wherein the administration reduces bone formation in the subject.
8. A pharmaceutical composition comprising denosumab for use in a method of inhibiting the development of new erosive joints in a subject in need thereof, wherein the method comprises administering to the subject denosumab in an amount equivalent to about 45 mg to about 60 mg (e.g., about 45 mg to less than 60 mg; about 45 mg to about 55 mg; about 45 mg to about 50 mg; about 45 mg; about 50 mg; about 55 mg; about 60 mg) denosumab administered once every about 3 months.
9. The pharmaceutical composition for use according to Clause 8, wherein the subject has been diagnosed with erosive hand osteoarthritis (OA).
10. The pharmaceutical composition for use according to any one of Clauses 1 to 9, wherein the administration inhibits the development of new erosive IP joints in the subject (e.g., at 48 weeks).
11. The pharmaceutical composition for use according to any one of Clauses 1 to 10, wherein the administration reduces the number of ‘S/J’ IP joints that develop ‘E’ phases in the subject.
12. The pharmaceutical composition for use according to any one of Clauses 1 to 11, wherein the administration inhibits the development of ‘E’ phases in ‘S/J’ IP joints in the subject.
13. A pharmaceutical composition comprising denosumab for use in a method of reducing pain in a subject with erosive hand osteoarthritis (OA), wherein the method comprises administering to the subject denosumab in an amount equivalent to about 45 mg to about 60 mg (e.g., about 45 mg to less than 60 mg; about 45 mg to about 55 mg; about 45 mg to about 50 mg; about 45 mg; about 50 mg; about 55 mg; about 60 mg) denosumab administered once every about 3 months.
14. The pharmaceutical composition for use according to any one of Clauses 1 to 13, wherein the administration reduces pain in the subject as assessed by numeric rating scale (NRS) pain.
15. The pharmaceutical composition for use according to any one of Clauses 1 to 14, wherein the administration improves erosive IP finger joint remodeling in the subject (e.g., at one year; at two years).
16. The pharmaceutical composition for use according to any one of Clauses 1 to 15, wherein the administration increases total GUSS™ in the subject (e.g., at 12 weeks; at 48 weeks).
17. The pharmaceutical composition for use according to any one of Clauses 1 to 16, wherein the administration improves joint function in the subject.
18. The pharmaceutical composition for use according to any one of Clauses 1 to 17, wherein the administration improves joint function in the subject as assessed by the Australian-Canadian Hand Osteoarthritis Index (AUSCAN).
19. The pharmaceutical composition for use according to any one of Clauses 1 to 18, wherein the administration improves joint function in the subject as assessed by the Functional Index for Hand Osteoarthritis (FIHOA).
20. The pharmaceutical composition for use according to any one of Clauses 1 to 19, wherein the subject does not exhibit a dose limiting toxicity (DLT) during denosumab administration.
21. The pharmaceutical composition for use according to any one of Clauses 1 to 20, wherein the subject does not exhibit any grade 3 or grade 4 adverse events associated with denosumab during denosumab administration.
22. The pharmaceutical composition for use according to any one of Clauses 1 to 21, wherein the subject is not hypocalcemic immediately prior to or during denosumab administration.
23. The pharmaceutical composition for use according to any one of Clauses 1 to 22, wherein the method comprises administering to the subject denosumab in an amount equivalent to about 45 mg to less than 60 mg denosumab administered once every about 3 months.
24. The pharmaceutical composition for use according to any one of Clauses 1 to 22, wherein the method comprises administering to the subject denosumab in an amount equivalent to about 45 mg denosumab administered once every about 3 months.
25. The pharmaceutical composition for use according to any one of Clauses 1 to 22, wherein the method comprises administering to the subject denosumab in an amount equivalent to about 50 mg denosumab administered once every about 3 months.
26. The pharmaceutical composition for use according to any one of Clauses 1 to 22, wherein the method comprises administering to the subject denosumab in an amount equivalent to about 55 mg denosumab administered once every about 3 months.
27. The pharmaceutical composition for use according to any one of Clauses 1 to 22, wherein the method comprises administering to the subject denosumab in an amount equivalent to about 60 mg denosumab administered once every about 3 months.
28 The pharmaceutical composition for use according to any one of Clauses 1 to 22, wherein the method comprises administering to the subject about 45 mg denosumab once every about 3 months.
29. The pharmaceutical composition for use according to any one of Clauses 1 to 22, wherein the method comprises administering to the subject about 50 mg denosumab once every about 3 months.
30. The pharmaceutical composition for use according to any one of Clauses 1 to 22, wherein the method comprises administering to the subject about 55 mg denosumab once every about 3 months.
31. The pharmaceutical composition for use according to any one of Clauses 1 to 22, wherein the method comprises administering to the subject about 60 mg denosumab once every about 3 months.
32. The pharmaceutical composition for use according to any one of Clauses 1 to 22, wherein the amount of denosumab provides:
   a. a mean serum concentration of denosumab substantially similar to that achieved by administering 60 mg denosumab once every about 3 months; and/or
   b. a mean plasma area under the curve (AUC_{0-∞}) substantially similar to that achieved by administering 60 mg denosumab once every about 3 months; and/or
   c. a difference between the mean plasma Cₘₐₓ at steady state and a mean plasma Cₘᵢₙ at steady state substantially similar to that achieved by administering 60 mg denosumab once every about 3 months; and/or
   d. a mean plasma Cₘₐₓ at steady state substantially similar to that achieved by administering denosumab 60 mg once every about 3 months.
33. The pharmaceutical composition for use according to any one of Clauses 1 to 22, wherein the amount of denosumab provides:
   a. a mean serum concentration of denosumab substantially similar to that achieved by administering 55 mg denosumab once every about 3 months; and/or
   b. a mean plasma area under the curve (AUC_{0-∞}) substantially similar to that achieved by administering 55 mg denosumab once every about 3 months; and/or
   c. a difference between the mean plasma Cmax at steady state and a mean plasma Cmin at steady state substantially similar to that achieved by administering 55 mg denosumab once every about 3 months; and/or
   d. a mean plasma Cmax at steady state substantially similar to that achieved by administering denosumab 55 mg once every about 3 months.
34. The pharmaceutical composition for use according to any one of Clauses 1 to 22, wherein the amount of denosumab provides:
   a. a mean serum concentration of denosumab substantially similar to that achieved by administering 50 mg denosumab once every about 3 months; and/or
   b. a mean plasma area under the curve (AUC0-∞) substantially similar to that achieved by administering 50 mg denosumab once every about 3 months; and/or
   c. a difference between the mean plasma Cmax at steady state and a mean plasma Cmin at steady state substantially similar to that achieved by administering 50 mg denosumab once every about 3 months; and/or
   d. a mean plasma Cmax at steady state substantially similar to that achieved by administering denosumab 50 mg once every about 3 months.
35. The pharmaceutical composition for use according to any one of Clauses 1 to 22, wherein the amount of denosumab provides:
   a. a mean serum concentration of denosumab substantially similar to that achieved by administering 45 mg denosumab once every about 3 months; and/or
   b. a mean plasma area under the curve (AUC0-∞) substantially similar to that achieved by administering 45 mg denosumab once every about 3 months; and/or
   c. a difference between the mean plasma Cmax at steady state and a mean plasma Cmin at steady state substantially similar to that achieved by administering 45 mg denosumab once every about 3 months; and/or
   d. a mean plasma Cmax at steady state substantially similar to that achieved by administering denosumab 45 mg once every about 3 months.
36. The pharmaceutical composition for use according to any one of Clauses 1 to 35, wherein the subject is administered denosumab for at least 12 weeks.
37. The pharmaceutical composition for use according to any one of Clauses 1 to 36, wherein the subject is administered denosumab for at least 24 weeks.
38. The pharmaceutical composition for use according to any one of Clauses 1 to 37, wherein the subject is administered denosumab for at least 48 weeks.
39. The pharmaceutical composition for use according to any one of Clauses 1 to 38, wherein the subject is administered denosumab for at least 96 weeks.
40. The pharmaceutical composition for use according to any one of Clauses 1 to 39, wherein the subject is administered denosumab once every about 3 months.
41. The pharmaceutical composition for use according to any one of Clauses 1 to 40, wherein the subject is administered denosumab once every about 3 months for at least 2 cycles (e.g., 2 cycles; 3 cycles; 4 cycles; 5 cycles; 6 cycles; 7 cycles; 8 cycles; at least 2 cycles; at least 3 cycles; at least 4 cycles; at least 5 cycles; at least 6 cycles; at least 7 cycles; at least 8 cycles).
42. The pharmaceutical composition for use according to any one of Clauses 1 to 41, wherein the pharmaceutical composition is adapted for administration to the subject by injection (e.g., by subcutaneous injection).
43. The pharmaceutical composition for use according to any one of Clauses 1 to 42, wherein the pharmaceutical composition is adapted for administration to the subject by injection (e.g., by subcutaneous injection) into the upper arm, upper thigh, or abdomen of the subject.
44. The pharmaceutical composition for use according to any one of Clauses 1 to 43, wherein the method further comprises administering to the subject at least one therapeutic agent chosen from analgesics and non-steroidal anti-inflammatory drugs.
45. The pharmaceutical composition for use according to any one of Clauses 1 to 44, wherein the method further comprises administering to the subject a stable dose of at least one therapeutic agent chosen from analgesics and non-steroidal anti-inflammatory drugs.
46. The pharmaceutical composition for use according to any one of Clauses 1 to 45, wherein the method further comprises administering to the subject daily supplements of oral calcium and vitamin D3 (e.g., daily supplements of oral calcium (1000 mg elemental calcium) and vitamin D₃ (880 international units)).
47. The pharmaceutical composition for use according to any one of Clauses 1 to 46, wherein the subject exhibits at least one sign of clinical or sonographic inflammation (e.g., in at least one interphalangeal finger joint) prior to denosumab administration.
48. The pharmaceutical composition for use according to any one of Clauses 1 to 47, wherein the subject exhibits at least one interphalangeal finger joint in the J or E phase with at least one inflammatory sign prior to denosumab administration.
49. The pharmaceutical composition for use according to any one of Clauses 1 to 48, wherein the subject exhibits at least one interphalangeal finger joint in the J or E phase with palpable swelling prior to denosumab administration.
50. The pharmaceutical composition for use according to any one of Clauses 1 to 49, wherein the subject is at least about 18 years old (e.g., at least about 30 years old).
51. The pharmaceutical composition for use according to any one of Clauses 1 to 50, wherein the subject has not been diagnosed with vitamin D deficiency, a chronic inflammatory rheumatic disease, psoriasis, cancer, or a chronic infectious disease.
52. The pharmaceutical composition for use according to any one of Clauses 1 to 51, wherein the subject has not been previously treated for erosive hand osteoarthritis.
53. The pharmaceutical composition for use according to any one of Clauses 1 to 51, wherein the subject was previously treated for erosive hand osteoarthritis.
54. The pharmaceutical composition for use according to Clause 53, wherein the subject was previously treated with at least one therapeutic agent chosen from tumor necrosis factor α (TNFα) blocking agents, anti-interleukin-1a inhibitors, anti-interleukin-1β inhibitors, and combinations thereof.
55. The pharmaceutical composition for use according to Clause 53 or 54, wherein the subject was previously treated with at least one therapeutic agent chosen from adalimumab, etanercept, lutikizumab, and combinations thereof.

### Agents Targeting RANK-L

Alternative aspects of the disclosure employ at least one agent targeting RANK-L in place of denosumab in a method, use, or pharmaceutical composition for use described herein. Agents targeting RANK-L include RANK-L antigen binding proteins (e.g., anti-RANK-L antibodies, antigen binding fragments thereof, and anti-RANK-L antibody protein products, some of which are described in International Patent Application Publication Nos. WO 2018/200918 and WO 03/002713 and U.S. Pat. No. 7,364,736, each of which is incorporated herein by reference in its entirety). In some agents, a RANK-L antigen binding protein that binds to human RANK-L, which has the amino acid sequence described in National Center for Biotechnology Information (NCBI) Reference Sequence No. NP003692, or SEQ ID NO: 1, and is encoded by the polynucleotide sequence of SEQ ID NO: 2, is employed in place of denosumab in a method, use, or pharmaceutical composition for use described herein.

In some embodiments, the RANK-L antigen binding protein is an anti-RANK-L antibody, or an antibody binding fragment thereof, or an anti-RANK-L antibody protein product.

In some embodiments, the anti-RANK-L antibody is a monoclonal antibody. Accordingly, in some embodiments, the present disclosure provides a liquid composition comprising a monoclonal antibody. Alternatively, in some embodiments, the liquid composition may be a polyclonal antibody composition. In some embodiments, the antibody comprises a sequence that is substantially similar to a naturally-occurring antibody produced by a mammal, e.g., mouse, rabbit, goat, horse, chicken, hamster, human, and the like. In this regard, the antibody can be considered as a mammalian antibody, e.g., a mouse antibody, rabbit antibody, goat antibody, horse antibody, chicken antibody, hamster antibody, human antibody, and the like. In some embodiments, the antibody is a human antibody. In some embodiments, the antibody is a chimeric antibody or a humanized antibody.

In some embodiments, the antibody is an anti-RANK-L antibody, e.g., an anti-RANK-L monoclonal antibody. In some embodiments, the anti-RANK-L antibody is an IgG2 antibody. In some embodiments, the anti-RANK-L antibody binds to human RANK-L, which, in some embodiments, comprises the amino acid sequence of SEQ ID NO: 1 and is encoded by the nucleotide sequence of SEQ ID NO: 2.

In some embodiments, the antibody is denosumab or a biosimilar thereof. Denosumab is known in the art. See, e.g., International Nonproprietary Names for Pharmaceutical Substances (INN): Proposed INN: List 56, WHO Drug Information 20 (3): 211 (2006); CAS Registry Number 615258-40-7. Denosumab is an immunoglobulin G2 (IgG2), also known as AMG 162, and is the active pharmaceutical ingredient of Prolia® and Xgeva®.

In some embodiments, the antibody comprises a light chain comprising a CDR1, CDR2, and CDR3 as set forth in Table 1. In some embodiments, the antibody comprises a heavy chain comprising a CDR1, CDR2, and CDR3 as set forth in Table 1. In some embodiments, the antibody comprises the VH and VL sequences recited in Table 1 or sequences comprising the VH-IgG2 and VL-IgG kappa sequences recited in Table 1. In some embodiments, the RANK-L antigen binding protein is an antibody comprising the amino acid sequences of SEQ ID NOs: 3-8. In some embodiments, the anti-RANK-L antibody comprises six CDR amino acid sequences of SEQ ID NOs: 3-8. In some embodiments, the anti-RANK-L antibody comprises a heavy chain (HC) complementarity-determining region (CDR) 1 amino acid sequence of SEQ ID NO: 3, an HC CDR2 amino acid sequence of SEQ ID NO: 4, an HC CDR3 amino acid sequence of SEQ ID NO: 5, a light chain (LC) CDR1 amino acid sequence of SEQ ID NO: 6, an LC CDR2 amino acid sequence of SEQ ID NO: 7, and an LC CDR3 amino acid sequence of SEQ ID NO: 8. In some embodiments, the anti-RANK-L antibody comprises a RANK-L-binding domain comprising (a) a heavy chain variable region (VH) that comprises: (i) a VH complementarity determining region one (CDR-H1) comprising the amino acid sequence of SEQ ID NO: 3; (ii) a CDR-H2 comprising the amino acid sequence of SEQ ID NO: 4; and (iii) a CDR-H3 comprising the amino acid sequence of SEQ ID NO: 5; and (b) a light chain variable region (VL) that comprises: (i) a VL complementarity determining region one (CDR-L1) comprising the amino acid sequence of SEQ ID NO: 6; (ii) a CDR-L2 comprising the amino acid sequence of SEQ ID NO: 7; and (iii) a CDR-L3 comprising the amino acid sequence of SEQ ID NO: 8. In some embodiments, the RANK-L-binding domain comprises: a VH that comprises the amino acid sequence of SEQ ID NO: 9, and a VL that comprises the amino acid sequence of SEQ ID NO: 10. In some embodiments, the anti-RANK-L antibody comprises a VH comprising the amino acid sequence of SEQ ID NO: 9 and a VL comprising the amino acid sequence of SEQ ID NO: 10. In some embodiments, the anti-RANK-L antibody comprises a HC comprising the amino acid sequence of SEQ ID NO: 11 or 13 and a LC comprising the amino acid sequence of SEQ ID NO: 12.

TABLE 1
Denosumab Amino Acid Sequences

| Description | Sequence | SEQ ID NO: |
|---|---|---|
| HC CDR1 | SYAMS | 3 |
| HC CDR2 | GITGSGGSTYYADSVK | 4 |
| HC CDR3 | DPGTTVIMSWFDP | 5 |
| LC CDR1 | RASQSVRGRYLA | 6 |
| LC CDR2 | GASSRAT | 7 |
| LC CDR3 | QQYGSSPRT | 8 |
| VH | EVQLLESGGGLVQPGGSLRLSCAASGFTFSSYAMSWVR | 9 |
| | QAPGKGLEWVSGITGSGGSTYYADSVKGRFTISRDNSK | |
| | NTLYLQMNSLRAEDTAVYYCAKDPGTTVIMSWFDPWG | |
| | QGTLVTVSS | |
| VL | EIVLTQSPGTLSLSPGERATLSCRASQSVRGRYLAWYQQ | 10 |
| | KPGQAPRLLIYGASSRATGIPDRFSGSGSGTDFTLTISRLE | |
| | PEDFAVFYCQQYGSSPRTFGQGTKVEIK | |
| VH-IgG2 | EVQLLESGGGLVQPGGSLRLSCAASGFTFSSYAMSWVR | 11 |
| | QAPGKGLEWVSGITGSGGSTYYADSVKGRFTISRDNSK | |
| | NTLYLQMNSLRAEDTAVYYCAKDPGTTVIMSWFDPWG | |
| | QGTLVTVSSASTKGPSVFPLAPCSRSTSESTAALGCLVK | |
| | DYFPEPVTVSWNSGALTSGVHTFPAVLQSSGLYSLSSVV | |
| | TVPSSNFGTQTYTCNVDHKPSNTKVDKTVERKCCVECP | |
| | PCPAPPVAGPSVFLFPPKPKDTLMISRTPEVTCVVVDVS | |
| | HEDPEVQFNWYVDGVEVHNAKTKPREEQFNSTFRVVS | |
| | VLTVVHQDWLNGKEYKCKVSNKGLPAPIEKTISKTKGQ | |
| | PREPQVYTLPPSREEMTKNQVSLTCLVKGFYPSDIAVEW | |
| | ESNGQPENNYKTTPPMLDSDGSFFLYSKLTVDKSRWQQ | |
| | GNVFSCSVMHEALHNHYTQKSLSLSP(GK)* | |
| VL-IgG | EIVLTQSPGTLSLSPGERATLSCRASQSVRGRYLAWYQQ | 12 |
| Kappa | KPGQAPRLLIYGASSRATGIPDRFSGSGSGTDFTLTISRLE | |
| | PEDFAVFYCQQYGSSPRTFGQGTKVEIKRTVAAPSVFIFP | |
| | PSDEQLKSGTASVVCLLNNFYPREAKVQWKVDNALQS | |
| | GNSQESVTEQDSKDSTYSLSSTLTLSKADYEKHKVYAC | |
| | EVTHQGLSSPVTKSFNRGEC | |

LC, light chain; HC, heavy chain; VL, variable light chain; VH, variable heavy chain.
* Terminal residues G and K may be clipped during recombinant production process.

In some embodiments, the antibody comprises:
i. a heavy chain (HC) CDR1 comprising an amino acid sequence of SEQ ID NO: 3 or an amino acid sequence which is at least 90% (e.g., at least 95%, at least 96%, at least 97%, at least 98%, or at least 99%) identical to SEQ ID NO: 3 or a variant amino acid sequence of SEQ ID NO: 3 with 1 or 2 amino acid substitutions;
ii. a HC CDR2 comprising an amino acid sequence of SEQ ID NO: 4 or an amino acid sequence which is at least 90% (e.g., at least 95%, at least 96%, at least 97%, at least 98%, or at least 99%) identical to SEQ ID NO: 4 or a variant amino acid sequence of SEQ ID NO: 4 with 1 or 2 amino acid substitutions;
iii. a HC CDR3 comprising an amino acid sequence of SEQ ID NO: 5 or an amino acid sequence which is at least 90% (e.g., at least 95%, at least 96%, at least 97%, at least 98%, or at least 99%) identical to SEQ ID NO: 5 or a variant amino acid sequence of SEQ ID NO: 5 with 1 or 2 amino acid substitutions.
iv. a light chain (LC) CDR1 comprising an amino acid sequence of SEQ ID NO: 6 or an amino acid sequence which is at least 90% (e.g., at least 95%, at least 96%, at least 97%, at least 98% or at least 99%) identical to SEQ ID NO: 6 or a variant amino acid sequence of SEQ ID NO: 6 with 1 or 2 amino acid substitutions,
v. a LC CDR2 comprising an amino acid sequence of SEQ ID NO: 7 or an amino acid sequence which is at least 90% (e.g., at least 95%, at least 96%, at least 97%, at least 98%, or at least 99%) identical to SEQ ID NO: 7 or a variant amino acid sequence of SEQ ID NO: 7 with 1 or 2 amino acid substitutions,
vi. a LC CDR3 comprising an amino acid sequence of SEQ ID NO: 8 or an amino acid sequence which is at least 90% (e.g., at least 95%, at least 96%, at least 97%, at least 98%, or at least 99%) identical to SEQ ID NO: 8 or a variant amino acid sequence of SEQ ID NO: 8 with 1 or 2 amino acid substitutions,

In some embodiments, the antibody comprises: a HC variable region comprising an amino acid sequence of SEQ ID NO: 9, an amino acid sequence which is at least 90% (e.g., at least 95%, at least 96%, at least 97%, at least 98%, or at least 99%) identical to SEQ ID NO: 9, or a variant amino acid sequence of SEQ ID NO: 9 with 1 to 10 (e.g., 1 to 9, 1 to 8, 1 to 7, 1 to 6, 1 to 5, 1 to 4, 1 to 3, 1 or 2) amino acid substitutions.

In some embodiments, the antibody comprises: a LC variable region comprising an amino acid sequence of SEQ ID NO: 10, an amino acid sequence which is at least 90% (e.g., at least 95%, at least 96%, at least 97%, at least 98%, or at least 99%) identical to SEQ ID NO: 10, or a variant amino acid sequence of SEQ ID NO: 10 with 1 to 10 (e.g., 1 to 9, 1 to 8, 1 to 7, 1 to 6, 1 to 5, 1 to 4, 1 to 3, 1 or 2) amino acid substitutions.

In some embodiments, the antibody comprises a heavy chain comprising an amino acid sequence of SEQ ID NO: 11, an amino acid sequence which is at least 90% (e.g., at least 95%, at least 96%, at least 97%, at least 98%, or at least 99%) identical to SEQ ID NO: 11, or a variant amino acid sequence of SEQ ID NO: 11 with 1 to 10 (e.g., 1 to 9, 1 to 8, 1 to 7, 1 to 6, 1 to 5, 1 to 4, 1 to 3, 1 or 2) amino acid substitutions.

In some embodiments, the terminal lysine may be absent. In some embodiments, the terminal lysine may be present. In some embodiments, the terminal glycine-lysine may be absent. In some embodiments, the terminal glycine-lysine may be present. C-terminal lysine clipping is a common phenomenon occurring during the bioproduction of monoclonal antibodies. Often, the lysine residue is removed via carboxypeptidase D (CpD), which results in generation of a mixture of antibody isoforms bearing zero or one C-terminal lysine residues on each heavy chain. Further, following C-terminal lysine cleavage, peptidylglycine α-amidating monooxygenase (PAM) catalyzes the hydroxylation of glycine and removal of the glyoxylate from the glycine residue, leaving an amidated C-terminal proline. Therefore, during recombinant production of a monoclonal antibody, the product is often a mixture of C-terminal processing variants, with heavy chain C-terminus ends at (amidated) proline, glycine, or lysine.

In some embodiments, the antibody comprises a light chain comprising an amino acid sequence of SEQ ID NO: 12, an amino acid sequence which is at least 90% (e.g., at least 95%, at least 96%, at least 97%, at least 98%, or at least 99%) identical to SEQ ID NO: 12, or a variant amino acid sequence of SEQ ID NO: 12 with 1 to 10 (e.g., 1 to 9, 1 to 8, 1 to 7, 1 to 6, 1 to 5, 1 to 4, 1 to 3, 1 or 2) amino acid substitutions.

In some embodiments, the amino acid substitution is a conservative amino acid substitution.

### Subjects, Erosive Hand Osteoarthritis, and Efficacy

In some embodiments of the presently disclosed methods, the subject is a human subject. In some embodiments, the human subject is about 18 years old or older. In some embodiments, the human subject is about 30 years or older.

In some embodiments, the subject meets at least one of the inclusion criteria of the clinical trial described in Example 1 of this application. In some embodiments, the subject has erosive hand osteoarthritis (erosive hand OA) or a predisposition toward the same. In some embodiments, the subject exhibits the presence of at least 1 IP joint in the J or E phase with inflammatory signs, defined clinically and by ultrasound.

In some embodiments, the subject was previously treated with at least one therapeutic agent chosen from tumor necrosis factor α (TNFα) blocking agents, anti-interleukin-la inhibitors, anti-interleukin-1β inhibitors, and combinations thereof. In some embodiments, the subject was previously treated with at least one therapeutic agent chosen from adalimumab, etanercept, lutikizumab, and combinations thereof.

### Dosing and Frequency

Alternative methods disclosed herein comprise administering to the subject a RANK-L antigen binding protein, e.g., an anti-RANK-L antibody, e.g., denosumab. In some embodiments, the method comprises administering about 45 mg to about 60 mg of the RANK-L antigen binding protein, e.g., the anti-RANK-L antibody, e.g., denosumab. Optionally, the method comprises administering about 45 mg, about 50 mg, about 55 mg, or about 60 mg the RANK-L antigen binding protein, e.g., the anti-RANK-L antibody, e.g. denosumab, to the subject. In some embodiments, the RANK-L antigen binding protein, e.g., the anti-RANK-L antibody, e.g., denosumab, is administered to the subject about once every about 3 months or about once every 80 days to about 95 days, e.g., once every 80, 81, 82, 83, 84, 85, 86, 87, 88, 89, 90, 91, 92, 93, 94, or 95 days. In some embodiments, the method comprises administering the subject about 45 mg denosumab once every about 3 months. In some embodiments, the method comprises administering the subject about 60 mg denosumab once every about 3 months.

In some embodiments, the method comprises administering an amount equivalent to about 45 mg to about 60 mg denosumab once every about 3 months. Optionally, the amount provides: (a) a mean serum concentration of denosumab substantially similar to that achieved by administering 60 mg denosumab once every about 3 months; (b) a mean plasma area under the curve (AUC_{0-∞}) substantially similar to that achieved by administering 60 mg denosumab once every about 3 months; (c) a difference between the mean plasma Cₘₐₓ at steady state and a mean plasma Cₘᵢₙ at steady state substantially similar to that achieved by administering 60 mg denosumab once every about 3 months; and/or (d) a mean plasma Cₘₐₓ at steady state substantially similar to that achieved by administering denosumab 60 mg once every about 3 months. Optionally, the amount provides: (a) a mean serum concentration of denosumab substantially similar to that achieved by administering 45 mg denosumab once every about 3 months; (b) a mean plasma area under the curve (AUC_{0-∞}) which is substantially similar to that achieved by administering 45 mg denosumab once every about 3 months; (c) a difference between the mean plasma Cₘₐₓ at steady state and a mean plasma Cₘᵢₙ at steady state which is substantially similar to that achieved by administering 45 mg denosumab once every about 3 months; and/or (d) a mean plasma Cₘₐₓ at steady state which is substantially similar to that achieved by administering denosumab 45 mg once every about 3 months.

In some embodiments, the method comprises administering to the subject denosumab for at least 24 weeks, at least 48 weeks, or at least 96 weeks, if not longer. In some embodiments, the method comprises administering to the subject denosumab for at least 1 year, 2 years, 3 years or longer. In some embodiments, the method comprises administering to the subject denosumab once every about 3 months for at least 2 cycles, at least 4 cycles, or at least 8 cycles, wherein each cycle comprises about 80 to about 95 days, e.g., 80, 81, 82, 83, 84, 85, 86, 87, 88, 89, 90, 91, 92, 93, 94, or 95 days.

### Routes of Administration and Pharmaceutical Compositions

The agent targeting RANK-L, e.g., anti-RANK-L antibody, e.g., denosumab, can be administered to the subject via any suitable route of administration. For example, the anti-RANK-L antibody (e.g., denosumab) can be administered to a subject via parenteral, nasal, oral, pulmonary, topical, vaginal, or rectal administration. The following discussion on routes of administration is merely provided to illustrate non-limiting example embodiments and should not be construed as limiting the scope of the disclosure in any way.

Formulations suitable for parenteral administration include, but are not limited to, aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term “parenteral” means not through the alimentary canal but by some other route such as subcutaneous, intramuscular, intraspinal, or intravenous. The anti-RANK-L antibody (e.g., denosumab) can be administered with a physiologically acceptable diluent in a pharmaceutical carrier, such as a sterile liquid or mixture of liquids, including water, saline, aqueous dextrose and related sugar solutions, an alcohol, such as, e.g., ethanol or hexadecyl alcohol, a glycol, such as, e.g., propylene glycol or polyethylene glycol, dimethylsulfoxide, glycerol, ketals, such as, e.g., 2,2-dimethyl-153-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as, e.g., a soap or a detergent, suspending agent, such as, e.g., pectin, carbomers, methylcellulose, hydroxypropylmethylcellulose, or carboxymethylcellulose, or emulsifying agents and other pharmaceutical adjuvants.

Oils which can be used in parenteral formulations include, but are not limited to, petroleum, animal, vegetable, and synthetic oils. Specific non-limiting examples of oils include peanut, soybean, sesame, cottonseed, corn, olive, petrolatum, and mineral. Suitable fatty acids for use in parenteral formulations include, but are not limited to, oleic acid, stearic acid, and isostearic acid. Ethyl oleate and isopropyl myristate are non-limiting examples of suitable fatty acid esters.

In some embodiments, the parenteral formulations contain from about 0.5% to about 25% by weight of the anti-RANK-L antibody (e.g., denosumab) in solution. Preservatives and buffers can be used. In order to minimize or eliminate irritation at the site of injection, such compositions can contain one or more nonionic surfactants having a hydrophile-lipophile balance (HLB) of from about 12 to about 17. In some embodiments, the quantity of surfactant in such formulations ranges from about 5% to about 15% by weight. Suitable surfactants include, but are not limited to, polyethylene glycol sorbitan fatty acid esters, such as, e.g., sorbitan monooleate and the high molecular weight adducts of ethylene oxide with a hydrophobic base, formed by the condensation of propylene oxide with propylene glycol. In some embodiments, the parenteral formulations are presented in unit-dose or multi-dose sealed containers, such as, e.g., ampoules and vials, and can be stored in a freeze-dried (lyophilized) condition requiring only the addition of the sterile liquid excipient, e.g., water, for injections, immediately prior to use. In some embodiments, extemporaneous injection solutions and suspensions are prepared from sterile powders, granules, and tablets.

Injectable formulations are in accordance with the present disclosure. The requirements for effective pharmaceutical carriers for injectable compositions are well-known to those of ordinary skill in the art (see, e.g., Pharmaceutics and Pharmacy Practice, J. B. Lippincott Company, Philadelphia, PA, Banker and Chalmers, eds., pages 238-250 (1982), and ASHP Handbook on Injectable Drugs, Toissel, 4th ed., pages 622-630 (1986)).

In some embodiments, the RANK-L antigen binding protein, e.g., the anti-RANK-L antibody, e.g., denosumab is administered to the subject by injection. In some embodiments, the RANK-L antigen binding protein, e.g., the anti-RANK-L antibody, e.g., denosumab, is administered to the subject by subcutaneous injection. In some embodiments, the RANK-L antigen binding protein, e.g., the anti-RANK-L antibody, e.g., denosumab, is administered to the upper arm, upper thigh, or abdomen of the subject. In some embodiments, the RANK-L antigen binding protein, e.g., the anti-RANK-L antibody, e.g., denosumab, is administered to the upper arm, upper thigh, or abdomen of the subject by injection. In some embodiments, the RANK-L antigen binding protein, e.g., the anti-RANK-L antibody, e.g., denosumab, is administered to the upper arm, upper thigh, or abdomen of the subject by subcutaneous injection.

### Safety

In some embodiments, the methods comprise administering an anti-RANK-L antibody (e.g., denosumab) in an amount that does not lead to a dose-limiting toxicity (DLT) during treatment with the anti-RANK-L antibody (e.g., denosumab). In some embodiments, the subject does not exhibit a DLT during the administration. In some embodiments, the subject does not exhibit any grade 3 or grade 4 adverse events associated with treatment with the anti-RANK-L antibody (e.g., denosumab) during the treatment period. In some embodiments, the subject does not exhibit any grade 3 or grade 4 adverse events associated with treatment with the anti-RANK-L antibody (e.g., denosumab) during the treatment period. In some embodiments, the treatment period is at least one month (e.g., 2 months, 3, months, 4 months, 5 months, 6 months, 7 months, 8 months, 9 months, 10 months, 11 months, 12 months, 1 year, 1.5 years, 2 years).

In some embodiments, the subject is not hypocalcemic. In some embodiments, the subject is not hypocalcemic during the administration of the anti-RANK-L antibody (e.g., denosumab). In some embodiments, the subject is not hypocalcemic prior to the administration of the anti-RANK-L antibody (e.g., denosumab).

The following examples are provided to further illustrate embodiments of the present disclosure and are not meant in any way to limit its scope.

### EXAMPLES

### Example 1. RANKL-Blockade for the Treatment of Erosive Osteoarthritis (OA) of Interphalangeal Finger Joints

This Example describes a monocentric, randomized, placebo-controlled, double-blind, phase 2 study in patients with erosive hand osteoarthritis (OA) (ClinicalTrials.gov Identified: NCT02771860; EudraCT Number 2015-003223-53).

### Methods

### Study Design and Participants

Patients were recruited from the rheumatology outpatient clinic of the Ghent University Hospital in Belgium. The study was approved by the local ethics committee and was conducted in accordance with Good Clinical Practice guidelines and the Declaration of Helsinki. All patients provided written informed consent.

Adult patients aged ≥30 years and diagnosed with erosive hand OA were considered eligible for the clinical study. Erosive hand OA was defined as radiographic evidence of ≥1 IP joint in J (i.e., loss of joint space) or E (i.e., subchondral erosion(s)) phase according to the Verbruggen and Veys anatomical phase scoring system (19).

Key inclusion criteria included:
Males and females ≥30 years of age;
Subjects with hand OA having suffered from transient inflammatory attacks of the interphalangeal finger joints characteristic for what has been termed ‘inflammatory’ or ‘erosive’ hand OA;
Subjects with hand OA showing inflammatory signs, either clinically or ultrasonographically, of the interphalangeal finger joints;
Subjects with hand OA in which at least 1 interphalangeal finger joint has the typical appearance on the X-rays of a ‘J’ or ‘E’ phase joint as defined by the criteria mentioned above;
Subjects with hand OA where at least 1 interphalangeal finger joint in the ‘J’ or ‘E’ phase presents a palpable swelling; and
Able and willing to give written informed consent and to comply with the requirements of the study protocol.

Radiographs were scored by a reader for eligibility.

Key exclusion criteria included:
Patients with known hypersensitivities to mammalian-derived drug preparations;
Patients with clinically significant hypersensitivity to any of the components of Prolia®;
Current and/or Prior treatment with any investigational agent within 90 days, or five half-lives of the product, whichever is longer;
Previous administration of denosumab from clinical trials or others (e.g., commercial use);
Vitamin D deficiency [25 (OH) vitamin D level <20 ng/ml (<49.9 nmol/L)].
Possibility of replenishment and re-screening;
Subjects with current hypo- or hypercalcemia (normal serum calcium levels: 8.5-10.5 mg/dl or 2.12-2.62 mmol/L);
Patients currently under bisphosphonate (BP) treatment or any use of oral BPs within 12 months of study enrollment or intravenous BPs or strontium ranelate within 5 years of study enrollment;
Prior use of any chondroprotective drug within 90 days, e.g., chondroitin sulfate, glucosamine, avocado-soybean unsaponifiables, tetracyclins, corticosteroids;
Prior use of any immunomodulating drug with possible effects on proinflammatory cytokine metabolism within 90 days, e.g., corticosteroids, methotrexate, sulfasalazine, leflunomide, D-Penicillin, anti-malarials, cytotoxic drugs, TNF blocking agents;
History of drug or alcohol abuse in the last year;
Patients suffering from chronic inflammatory rheumatic disease (e.g., rheumatoid arthritis, spondylarthropathy, psoriatic arthritis, gout, chondrocalcinosis or other auto-immune diseases, e.g., systemic lupus erythematosus);
History of cancer or lymphoproliferative disease other than a successfully and completely treated squamous cell or basal cell carcinoma of the skin or cervical dysplasia, with no recurrence within the last two years;
History of any Solid Organ or Bone Marrow Transplant;
Comorbidities: significant renal function impairment (glomerular filtration <30 ml/min/1.73 m² or <50% of normal value), uncontrolled diabetes, unstable ischemic heart disease, congestive heart failure (NYHA III, IV), uncontrolled hypo or hyperparathyroidism, active inflammatory bowel disease, malabsorption, liver failure or chronic hepatic disease (serum AST/ALT levels 3 times above normal), recent stroke (within three months), chronic leg ulcer and any other condition (e.g., indwelling urinary catheter) which, in the opinion of the investigator, would put the subject at risk by participation in the protocol;
Subject has any kind of disorder that compromises the ability of the subject to give written informed consent and/or to comply with study procedures;
Patient who is pregnant or planning pregnancy; if the female subject is of child-bearing age, she must use a valid mean of contraception during the study and for 9 months after last dose of study medication. For males with a partner of childbearing potential: subject refuses to use 1 effective methods of contraception for the duration of the study and for 10 months after the last dose of study medication;
Female subjects who are breast-feeding;
History of osteonecrosis of the jaw, and/or recent (within 3 months) tooth extraction or other unhealed dental surgery; or planned invasive dental work during the study.

### Randomization and Masking

After screening, patients were randomly assigned in a 1:1 ratio to placebo or denosumab 60 mg subcutaneously every 12 weeks for 48 weeks by use of a block randomization scheme with a fixed block size of four. The randomization list was generated by a co-worker independent of the study and not involved in any procedure during the study. Study medication was provided by the pharmacy department. Medication and placebo syringes were identical. Patients, outcome assessors, and data analyst remained masked for treatment allocation until lock of the study database.

### Procedures

Study medication was administered on site by a dedicated nurse or physician at baseline, weeks 12, 24, and 36 of the placebo-controlled phase, and in the open label extension period at weeks 48, 72, and 84. All patients received daily supplements of oral calcium (1000 mg elemental calcium) and vitamin D₃ (880 international units). All medication intake, use of rescue medication or changes in concomitant medication was registered throughout the entire study. Patients were allowed to take stable doses of analgesics and/or non-steroidal anti-inflammatory drugs as rescue medication. Intake of corticosteroids was not allowed.

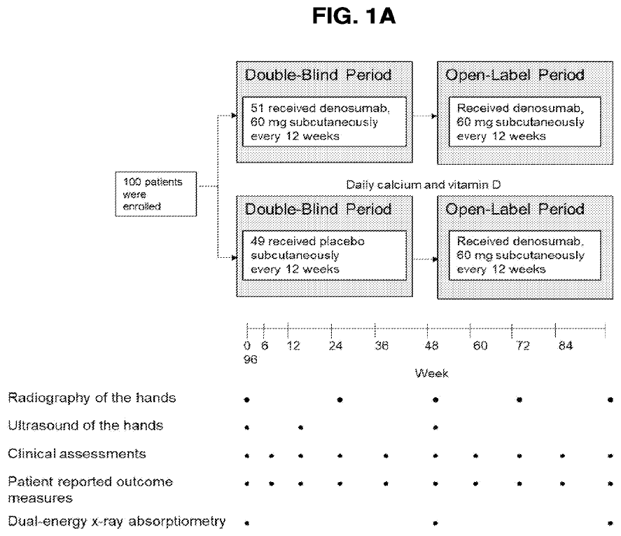

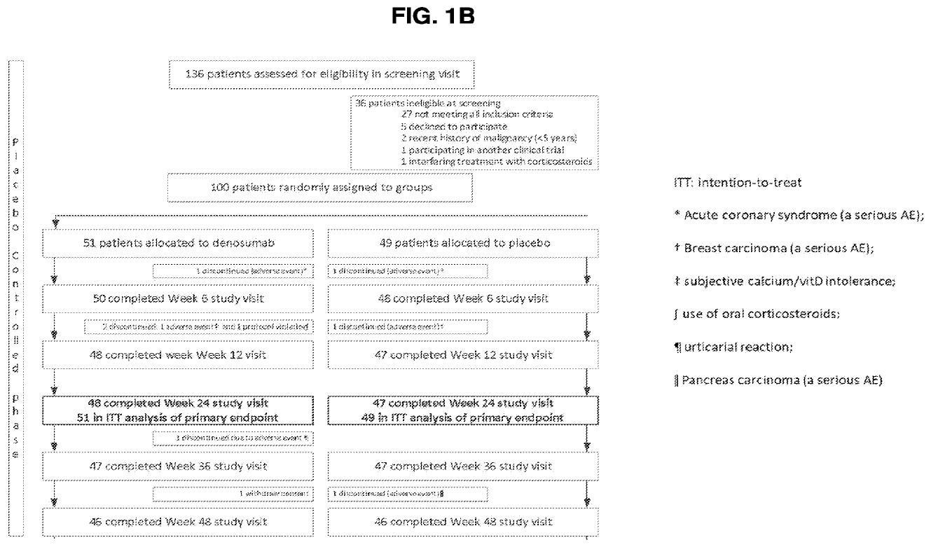

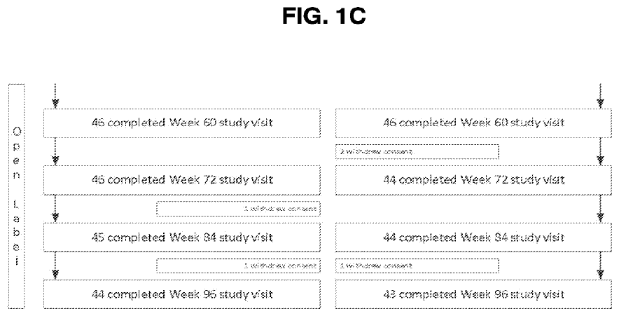

Hand radiographs were taken at baseline and weeks 24, 48, 72, and 96 (FIG. 1A). Ultrasonography (US) was performed by an experienced sonographer with more than 10 years of experience, at baseline and weeks 12 and 48. Synovial proliferation (0-3), effusion (0-3), Power Doppler (PD) signal (0-3), and erosions (present/absent) in proximal IP (PIP) and distal IP (DIP) joints 2-5 were recorded. Clinical efficacy endpoints assessed by the same trained physician, blinded for US results, through week 96 included the absence or presence of tenderness upon palpation and soft tissue swelling of PIP and DIP joints 2-5, and grip strength (in kg).

Patient-reported endpoints through week 96 included numeric rating scale (NRS) pain (0-10), the Australian-Canadian Hand Osteoarthritis Index (AUSCAN) (0-150), the Functional Index for Hand Osteoarthritis (FIHOA) (0-30), and a patient global assessment of efficacy on NRS (0-10).

Bone mineral density was measured by dual-energy x-ray absorptiometry at baseline and weeks 48 and 96.

### Radiographic Assessments

Baseline radiographic status and changes in subchondral plate, subchondral bone, and joint space were measured in all PIP and DIP joints 2-5 according to the Ghent University Scoring System (GUSS™) (13). This scoring system is composed of 3 subdomains: subchondral plate; subchondral bone; and joint space. Specific features referring to the underlying pathology of the disease are scored on a numerical scale from 0 to 100, with increments of 10. The maximum score refers to a normal or completely restored (i.e., non-erosive) joint. A total score per joint is made by an equally weighted sum score of all 3 subdomains (minimum 0); maximum 300). Details of GUSS™ are described elsewhere and an educational atlas is available for consultation (13). Thus, the scoring system can change in positive (i.e., more remodeling) or negative direction (i.e., more erosive progression).

The presence of anatomical phases were assessed by the Verbruggen and Veys scoring system on baseline and weeks 24, 48, 72, and 96 radiographs (19). This categorical scoring system differentiates normal joints (N) from pre-erosive phases (S phase, i.e., stationary phase with minimal degenerative features such as subchondral sclerosis, joint spacc narrowing, and presence of small osteophytes, and J phase with partial or complete loss of joint space), erosive phase (E), and phases of remodeling (R, i.e., signs of repair such as reappearance of subchondral plate and joint space width, disappearance of erosions at the subchondral bone and development of osteophytes at joint margins, and F, fused joint as extreme sign of remodeling).

Target joints were defined as IP joints in J or E phase on baseline radiographs with presence of inflammatory activity, defined by clinical soft tissue swelling and ultrasonographic inflammation (i.e., either synovial proliferation or effusion). If several target joints were available, all were included for efficacy analysis.

All radiographs were read independently by two trained assessors, blinded to randomization and clinical data. The radiographs were read with knowledge of time sequence. Interreader reliability analysis was performed on the readings for each 20 patients. Intraclass coefficients of correlation (ICCs) and weighted kappa (κ) statistics were calculated for GUSS™ scores and anatomical phases, respectively. If these did not exceed 0.80, retraining was performed for the following 20 patients until κ>0.80. Intrareader reliability was studied by repeated readings of a series of images (first 20 patients) by both readers with an interval of minimally one month. The final scores of radiographic scorings were the agreement scores amongst the two readers. In case of no absolute agreement, a consensus score was made.

### Outcomes

The primary efficacy endpoint was the change in total GUSS™ at week 24.

Secondary radiographic endpoints were the total GUSS™ changes from baseline at week 48, and the percentage of new erosive joints (J/E) by Verbruggen and Veys amongst the baseline pre-erosive joints (i.e., baseline N, S, J joints) per patient at weeks 24 and 48. Exploratory clinical endpoints were NRS pain at week 24, FIHOA, AUSCAN, and grip strength, tender joints count (0-16), and clinical swollen joint count (0-16) at week 24. Other imaging endpoints were US synovitis score (0-96), US synovial proliferation score (0-48), US effusion score (0-48), US PD score (0-48) at week 12, and US erosion score (0-16) at week 48, and percentage changes from baseline in bone mineral density at femoral neck and lumbar spine at week 48 and 96.

Safety endpoints included the number of (serious) adverse events, withdrawal because of adverse events, and changes in laboratory data throughout the study.

### Statistical Analyses

A sample size of 46 patients in each treatment arm was required to detect a difference in mean change GUSS™ of 20 units between the placebo and treated group at week 24 attaining a power of 90%, assuming that the standard deviation (SD) was 29 using a t-test with a two-sided 0.05 level of significance (α). Taking into account an attrition rate of 8%, a total of 100 patients was included in the study.

Primary efficacy analyses were performed in an intention-to-treat (ITT) approach (all participants randomly assigned to groups and who attended a baseline visit). Changes in GUSS™ were analyzed at joint level with generalized estimating equations (GEE), accounting for within-patient clustering. Robust standard errors were used, and the working correlation structure specified exchangeable. The independent variables included in the model were treatment group, visit number (categorical), interaction between treatment group and visit number, and the baseline value of the dependent variable (continuous). Missing data were not imputed for descriptive statistics. For analysis of the primary endpoint, data available from all time points were used in each analysis, accounting for within patient clustering effects by using GEE models. Missing values after the last available visit or questionnaire or assessment due to dropping out the study for any reason were imputed according to a predefined imputation model. As there was only one primary outcome, no adjustments for multiple testing were performed. A prespecified sensitivity analysis for the primary endpoint was performed without imputation of missing data and with correction for baseline GUSS™ values.

For the primary outcome measure, a subgroup analysis for inflammatory activity in the joint (Yes/No) was done. The interaction between the presence of baseline inflammation and treatment effect on change in GUSS™ scores over 24 weeks was tested. For the primary outcome measure, another descriptive efficacy analysis was performed on an extended group of target joints (all joints showing any progression to J, E or E/R phase throughout the study that were not defined J or E at baseline).

Secondary imaging and exploratory outcomes were done in the per protocol population (all patients meeting the study entry criteria, with no major protocol deviations, who completed the study) and measured at the level of the patient (except for total GUSS™ at week 48). The GEE model for continuous data was similar to the model for the primary endpoint. For the binary outcome measures (for example E joint yes/no) a GEE logistic regression for grouped binomial data was applied. Primary and secondary efficacy analyses were presented by a point estimate of the difference between the treatment groups, or Odds ratios (OR) with a 95% confidence interval (95% CI) and the two-sided p-value. A p-value below 0.05 (p<0.05) was considered statistically significant.

For the analyses from year 2, similar GEE logistic regression models were used with treatment groups based on the initial randomization code from year 1 (i.e., patients having received denosumab during 1 year vs. 2 years). All statistical calculations were performed using R version 3.6.1 or IBM SPSS Statistics for Windows, version 25 (N.Y., USA).

### Results

### Subjects and Target Joints

Patients were screened for enrollment between March 2016 and July 2018. 136 patients were screened, 100 were randomized and received at least one administration of the study medication (FIG. 1B). The most common reason for exclusion was not meeting one or more of the inclusion criteria (most often absence of radiographic eligible joint(s) with soft tissue swelling). Of treated subjects, 49 were assigned to placebo (49%) and 51 to denosumab (51%) and were included in the ITT analysis of the primary endpoint. 46 patients in the denosumab group and 46 in the placebo group completed the 48-week study. Five patients in the denosumab (9.8%) (1 because of SAE, 3 due to withdrawal of consent, and 1 because of protocol deviation (use of corticosteroids)) group and 3 (6.1%) in the placebo group (all because of SAE) discontinued from the study. Demographic and baseline characteristics were well balanced between the groups (Table 2). The mean number of radiographically affected joints per patient was 3.6 and 4.0 in denosumab and placebo groups, respectively (p>0.05). 168 target joints were selected for ITT analysis of the primary outcome. All joints (n=1590) were analyzed for secondary imaging endpoint. Ten joints were missing due to amputation (n=9) or prosthesis (n=1). All patients were included for secondary clinical endpoints in the per protocol analysis.

TABLE 2
Demographic and clinical characteristics of the patients at baseline

| Characteristics | Denosumab (n = 51) | | Placebo (n = 49) | |
|---|---|---|---|---|
| Age - yr | 62.0 | (7.7) | 60.6 | (7.9) |
| Female sex - no. (%) | 41 | (80) | 37 | (76) |
| Disease duration - yr | 6.3 | (6.6) | 6.0 | (6.4) |
| Body-mass index † | 25.3 | (3.5) | 25.3 | (4.0) |
| NRS pain ‡ | 4.7 | (2.5) | 4.8 | (2.7) |
| AUSCAN∫ | 67.0 | (4.9) | 68.9 | (5.5) |
| FIHOA¶ | 10.4 | (0.9) | 10.3 | (1.0) |
| Mean GUSS ™ (of 16 joints)∥ | 249 | (66) | 248 | (67) |
| Anatomical phase according | | | | |
| to Verbruggen and Veys - | | | | |
| No. (%)** | | | | |
| N joints | 196 | (24.3) | 150 | (19.2) |
| S joints | 326 | (40.4) | 353 | (45.1) |
| J joints | 67 | (8.3) | 82 | (10.5) |
| E joints | 98 | (13.3) | 104 | (13.3) |
| R joints | 107 | (13.3) | 91 | (11.6) |
| F joints | 3 | (0.4) | 3 | (0.4) |
| Number of affected joints | 3.6 | (2.2) | 4.0 | (2.2) |
| (of 16 joints) †† | | | | |

† The body-mass index is the weight in kilograms divided by the square of the height in meters.
‡ The numeric rating scale (NRS) pain is a scale from 0 to 10, with higher scores indicating greater severity.
∫Scores of the Australian-Canadian Hand Osteoarthritis Index (AUSCAN) range from 0 to 150, with higher scores indicating more disability.
¶Scores of the Functional Index for Hand Osteoarthritis (FIHOA) range from 0 to 30, with higher scores indicating more disability.
∥The Ghent University scoring system (GUSS ™) range from 0 to 300, with higher scores indication remodeling or repair. Lower scores indicate greater erosions, loss of joint space or subchondral plate (13). Mean GUSS ™ value of 16 joints per patient is shown.
**The Verbruggen and Veys anatomical score system categorizes joints as normal (N), stationary (S, i.e. showing minimal signs of degeneration), partial or complete loss of joint space (J), erosive (E), remodeled (R), or fused (F, i.e. extreme repair) (19).
†† Any radiographically defined S, J, E, R joint, according to the Verbruggen and Veys score.

### Primary Radiographic Efficacy Endpoint

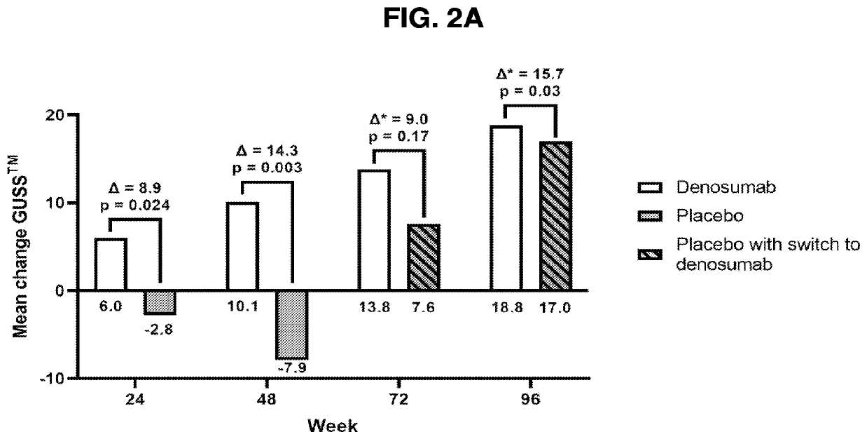

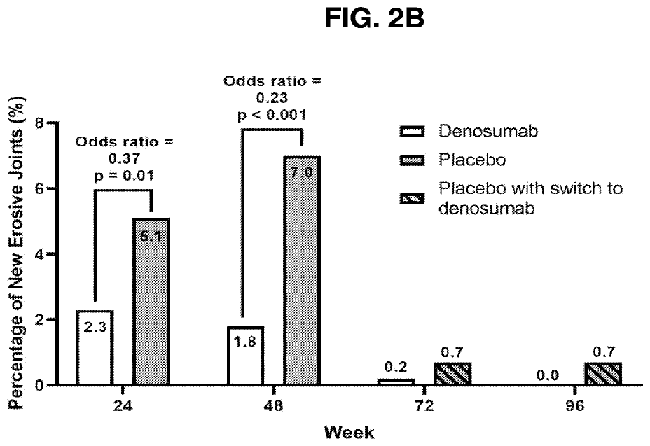

Change in total GUSS™ was found to be statistically higher in the denosumab group compared to the placebo group at week 24, indicating more remodeling and less erosive progression (estimated mean difference total GUSS™=8.9 (95% CI: 1.0 to 16.9; p=0.024)) (FIG. 2A). This effect was confirmed at week 48 (estimated mean difference total GUSS™=14.3 (95% CI: 4.6 to 24.0; p=0.003)). Inter- and intra-reader reliability data were excellent for all radiographic scores (Table 3).

TABLE 3
Inter- and intra-reader reliability analyses of radiographic scores by
GUSS ™ and anatomical scoring system by Verbruggen and Veys:
Baseline and follow up radiographs of the first 20 patients (n = 320 joints)

| | VV* | GUSS ™ | | | | | | |
|---|---|---|---|---|---|---|---|---|
| | Baseline data | | | | | Change scores | | |
| | | SC | Joint | SC | Total | Δ Total | Δ Total | Δ Total |
| | | plate | Width | Bone | score | score | score | score |
| Readers | | W 0 | W 0 | W 0 | W 0 | W 0-W 24 | W 0-W 48 | W 24-W 48 |

Intrareader reliability

| | | | | | | | | |
|---|---|---|---|---|---|---|---|---|
| Reader 1 | 0.917 | 0.938 | 0.958 | 0.979 | 0.980 | 0.822 | 0.893 | 0.802 |
| Reader 2 | 0.950 | 0.915 | 0.923 | 0.968 | 0.952 | 0.866 | 0.911 | 0.815 |

Interreader reliability

| | | | | | | | | |
|---|---|---|---|---|---|---|---|---|
| Reader 1 vs. | 0.925 | 0.998 | 1.0 | 0.943 | 0.988 | 0.991 | 0.994 | 0.993 |
| Reader 2 | | | | | | | | |

Inter- and intrareader reliability analyses of the radiographic scores by two radiographic scoring system, i.e., GUSS ™ and the anatomical scoring system by Verbruggen and Veys. Scores of subdomains are shown for baseline data, and change of the total scores for longitudinal data. Data shown are intra-class coefficients of correlation (ICC) by two-way mixed, absolute agreement, average measures, or stated if otherwise for GUSS ™ from the first 20 patients (accounting for 320 joints).
*Weighted kappa statistics from baseline data shown.
VV: anatomical phase scoring system by Verbruggen and Veys;
SC: subchondral;
GUSS ™: Ghent University scoring system;
Δ: change;
W: week

### Secondary Imaging Efficacy Endpoint

At the patient level, the development of new erosive joints was statistically higher in the placebo group compared to the denosumab group at week 48 (7.0% new E joints in placebo vs. 1.8% in denosumab) (FIG. 2B). From baseline to week 48, the estimated odds ratio for erosive progression was 76.7% lower in the denosumab group compared to the placebo group (OR=0.23 (95% CI: 0.11 to 0.50); p<0.001).

### Exploratory Imaging Endpoints

Both US effusion and US synovitis scores at week 12 decreased significantly in the denosumab and placebo groups at week 12 and week 48 compared to basclinc. The US erosion score was reduced significantly in the denosumab group at week 48 compared to baseline; this was not the case in the placebo group. However, the adjusted mean between group difference from baseline to week 48 was not found statistically significant (Table 4).

In this non-osteoporotic population, the mean bone mineral density T scores at the lumbar spine and femoral neck increased consistently from baseline through to week 96 in the denosumab-treated group. Also, in the placebo group, improvement was seen at the lumbar spine at week 48 but not at the femoral neck. At 48 weeks, the percentage change from baseline was greater with denosumab treatment than with placebo at the lumbar spine by 2.8 percentage points (p<0.001) (Table 5).

Sensitivity analyses without imputations and with correction for baseline GUSS™ measures, and Per protocol analysis of the primary endpoint showed similar results as in the ITT (data not shown).

The interaction between the presence of baseline inflammation (yes/no) and treatment effect on change in GUSS™ scores was tested and showed no significant interaction between inflammation and treatment at week 24 (p=0.48) or week 48 (p=0.18).

TABLE 4
Changes in ultrasonographic features between the two treatment groups

| | | | | | | | | P-value | P-value | | |
|---|---|---|---|---|---|---|---|---|---|---|---|
| | | | | | | | | Baseline | Baseline | Mean | |
| | Group | Baseline | | Week 12 | | Week 48 | | vs. W 12 | vs. W 48 | changes* | P-value† |
| Effusion | Denosumab | 13.61 | (6.55) | 10.04 | (5.50) | 11.52 | (6.11) | 0.001 | 0.024 | 0.781 | 0.583 |
| (0-3) | Placebo | 15.02 | (8.32) | 12.28 | (6.53) | 11.48 | (5.51) | 0.004 | 0.002 | | |
| Synovial | Denosumab | 11.18 | (5.96) | 10.76 | (6.62) | 10.67 | (6.22) | 0.815 | 0.772 | −0.587 | 0.640 |
| proliferation | Placebo | 11.55 | (5.41) | 10.81 | (5.48) | 12.57 | (7.79) | 0.398 | 0.434 | | |
| (0-3) | | | | | | | | | | | |
| Synovitis | Denosumab | 24.78 | (8.69) | 20.80 | (9.06) | 22.20 | (10.20) | 0.004 | 0.042 | 0.194 | 0.915 |
| (0-3) | Placebo | 26.57 | (11.28) | 23.09 | (8.24) | 24.04 | (10.76) | 0.004 | 0.025 | | |
| PD | Denosumab | 2.67 | (2.73) | 2.16 | (2.72) | 3.02 | (2.96) | 0.229 | 0.513 | −0.711 | 0.323 |
| (0-3) | Placebo | 3.18 | (6.17) | 2.02 | (3.17) | 2.65 | (3.04) | 0.056 | 0.534 | | |
| Erosions | Denosumab | 5.84 | (3.15) | 5.36 | (3.06) | 4.65 | (2.97) | 0.171 | 0.002 | 0.925^{∫} | 0.158^{∫} |
| (0-1) | Placebo | 6.65 | (3.12) | 6.21 | (3.29) | 6.37 | (3.24) | 0.444 | 0.187 | | |

Data are expressed as mean (standard deviation). Denosumab (N = 51) and placebo (N = 49) treated patients were included in the analysis. Significant p-values in bold (α = 0.05)
*Adjusted mean changes between groups over 12 weeks, adjusted for baseline values
†Comparing changes between the two groups over 12 weeks using independent samples t-test.
^{∫}change between baseline and week 48 for erosion.

TABLE 5
Bone Mineral Density at baseline and follow-up and percentage change from baseline

| | | | | | % change | % change | P- | P- |
|---|---|---|---|---|---|---|---|---|
| | | | | | Week 48 | Week 96 | value | value |
| | | T score | | | from | from | Baseline | between |
| | Group | Baseline | Week 48 | Week 96 | Baseline | Baseline | vs. W 48 | groups* |
| T score | Denosumab | −0.89 (0.96) | −0.83 (0.93) | −0.73 (0.94) | 0.29 (5.88) | 2.07 (6.18) | 0.113 | 0.504 |
| Femoral | | [−3.30; 2.00] | [−3.10; 2.00] | [−3.00; 2.00] | | | | |
| Neck | Placebo | −0.70 (1.10) | −0.74 (1.08) | −0.58 (0.96) | 0.35 (3.89) | 1.87 (3.88) | 0.574 | — |
| | | [−2.80; 1.80] | [−2.80; 1.90] | [−2.60; 1.70] | | | | |
| T score | Denosumab | −0.58 (1.22) | −0.28 (1.20) | −0.18 (1.21) | 4.41 (3.06) | 6.65 (3.29) | 0.000 | <0.001 |
| Lumbar | | [−3.30; 1.90] | [−3.10; 2.40] | [−3.30; 2.50] | | | | |
| Spine | Placebo | −0.61 (1.40) | −0.52 (1.36) | −0.63 (1.34) | 1.66 (3.01) | 5.47 (4.02) | 0.001 | — |
| | | [−4.00; 2.30] | [−3.60; 2.30] | [−2.10; 3.00] | | | | |
| T score | Denosumab | −0.87 (0.97) | −0.78 (1.00) | −0.65 (0.98) | 0.64 (3.28) | 1.67 (2.73) | 0.259 | 0.050 |
| ⅓^{rd} | | [−3.30; 1.00] | [−3.20; 1.30] | [−3.10; 1.20 | | | | |
| Distal | Placebo | −0.80 (1.09) | −0.96 (1.03) | −0.88 (0.96) | −0.55 (2.66) | −0.24 (2.89) | 0.090 | — |
| Radius | | [−3.0; 2.10] | [−3.10; 1.10] | [−2.80; 1.20] | | | | |

Data shown are mean (standard deviation)[range] unless otherwise stated; Denosumab (N = 48) and placebo (N = 47) were included in the analysis at week 48.
*Comparing changes between the two groups over 48 weeks using independent samples t-test. Significant p-values in bold (α = 0.05)

A descriptive efficacy analysis performed on an extended group of target joints (n=198) (i.e., all joints showing any progression to J, E or E/R phase throughout the study that were not defined J or E phase at baseline) showed a mean change in GUSS™ of 11.8 (95% CI=3.6 to 20.0) higher in denosumab compared to placebo (p=0.004) at week 24 and a change of 19.7 (95% CI=9.4 to 29.9) in favor of denosumab treatment (p<0.001) at week 48.

### Exploratory Clinical Endpoints

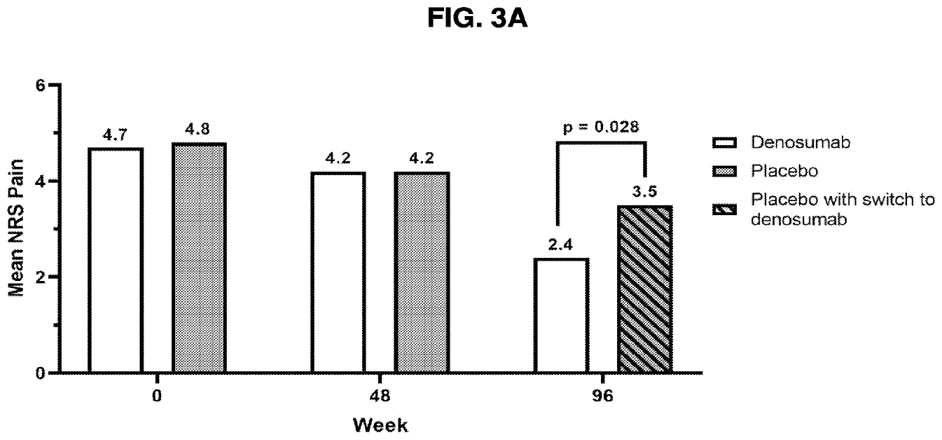

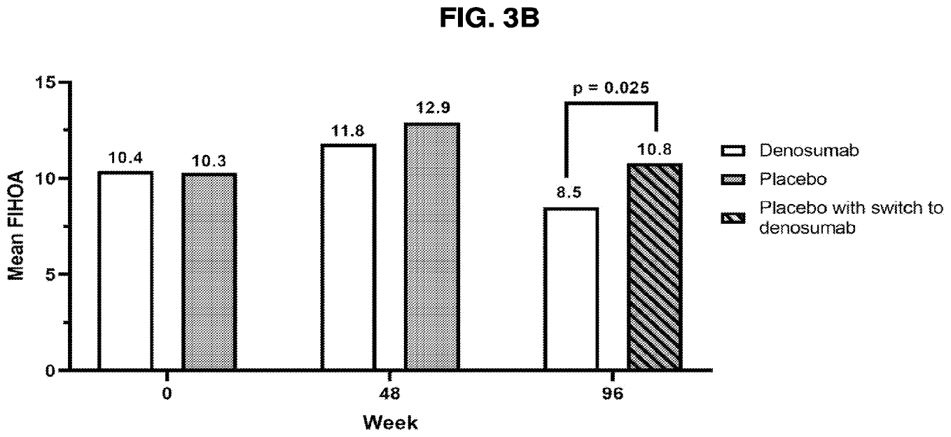

The change in pain (NRS) at week 24 versus baseline did not differ significantly between placebo and denosumab (−0.2 (95% CI: −1.0 to 0.6); p=0.68) (FIG. 3A). AUSCAN total and FIHOA were respectively −2.8 (95% CI: −11.8 to 6.2) and −1.2 (95% CI: −3.1 to 0.7) (FIG. 3B) lower in the denosumab group compared to the placebo group at week 24 without statistical significance being reached. NRS patient global opinion of efficacy was estimated to be 0.5 (95% CI: −0.7 to 1.8) higher in the denosumab group compared to the placebo group at week 24 (p=0.41). At week 48, the numerical difference in function (both AUSCAN and FIHOA) improved numerically but not statistically in the denosumab group compared to placebo (Tables 6, 7).

### Extension Phase

Ninety-two patients entered the 1-year extension phase at week 48, of which 46 originally received denosumab and 46 originally received placebo during the first year. Five patients prematurely discontinued during the open-label extension phase.

Comparable to year 1, target joints evolved toward remodeling in the second year. Total GUSS™ kept increasing in both groups during the second year compared to baseline, with a larger increase in the former placebo group at week 96 compared to the initial denosumab group (estimated mean difference total GUSS™ at week 72=2.3 (95% CI: −2.9 to 6.9; p=0.32) and estimated mean difference total GUSS™ at week 96=3.5 (95% CI: −1.1 to 8.1; p=0.13)). Compared to week 48, the change in GUSS™ score significantly increased at week 96 in the former placebo group (estimated mean difference total GUSS™ placebo=25.7 (95% CI: 16.2 to 35.1) vs. estimated mean difference total GUSS™ denosumab=9.9 (95% CI: −1.3 to 21.1); p=0.035). When including 18 newly developed target joints in the placebo group during the first year for the analysis at week 48, similar results were confirmed (estimated mean difference total GUSS™ placebo=27.0 (95% CI: 17.9 to 36.0) vs. estimated mean difference total GUSS™ denosumab=10.1 (95% CI: −0.4 to 20.6); p=0.017). Only three new erosive joints developed during the extension phase: 2 joints in J phase in two former placebo treated patients; and one in E phase (coming from J in year 1) in a denosumab-treated patient. Concerning the clinical exploratory endpoints, patients who initially received denosumab during the first year showed a significant decrease in pain levels at week 96, compared to baseline and compared to the patients from the initial placebo group (NRS pain denosumab week 96=2.42 vs. NRS pain denosumab baseline=4.68, p<0.001, and NRS pain denosumab week 96=2.42 vs. NRS pain initial placebo week 96=3.52, p=0.028, respectively) (FIG. 3A). Similar observations were seen for FIHOA (mean difference within denosumab group week 96 vs. baseline, p=0.042; mean difference between groups week 96, p=0.025) but not AUSCAN, suggesting that clinical benefits were induced only after two years of treatment with denosumab (Tables 6, 7).

TABLE 6
Clinical data from the placebo-controlled and open-label extension phase at Week 24

| | | | | P- | P- | | | P- | P- |
|---|---|---|---|---|---|---|---|---|---|
| | | | | value | value | | | value | value |
| | | | | within | between | | | within | between |
| | | | | groups vs. | groups vs. | | | groups vs. | groups vs. |
| Endpoint | Group | Week 24 | | baseline | baseline | Week 48 | | baseline | baseline |
| Change in NRS | Denosumab | −0.8 | (−17.3) | 0.018 | 0.690 | −0.5 | (−10.5) | 0.002 | 0.299 |
| pain (%) | Placebo | −0.7 | (−14.0) | 0.011 | — | −0.6 | (−12.5) | 0.035 | — |
| Change in FIHOA | Denosumab | 1.3 | (12.7) | 0.049 | 0.202 | 1.4 | (13.5) | 0.045 | 0.092 |
| (%) | Placebo | 2.3 | (22.7) | 0.001 | — | 2.6 | (25.3) | <0.001 | — |
| Change in | Denosumab | −3.8 | (−5.6) | 0.318 | 0.714 | −2.1 | (−3.1) | 0.597 | 0.567 |
| AUSCAN (%) | Placebo | −1.5 | (−2.2) | 0.766 | — | 1.0 | (1.5) | 0.621 | — |
| Change in tender | Denosumab | −0.3 | (−5.3) | 0.855 | 0.971 | −0.9 | (−17.0) | 0.154 | 0.563 |
| joint count (%) | Placebo | −0.6 | (−11.2) | 0.971 | — | −0.8 | (−14.0) | 0.375 | — |
| Change in swollen | Denosumab | −0.3 | (−7.9) | 0.432 | 0.255 | −0.1 | (−1.9) | 0.886 | 0.135 |
| joint count (%) | Placebo | −1.0 | (−20.3) | 0.016 | — | −1.0 | (−21.8) | 0.011 | — |
| Change in grip | Denosumab | 0.4 | (2.3) | 0.519 | 0.754 | 0.7 | (4.4) | 0.212 | 0.485 |
| strength (%) | Placebo | −0.2 | (−1.0) | 0.787 | — | −1.0 | (−5.4) | 0.451 | — |

Longitudinal changes compared to baseline are given and percentage changes compared to baseline. Comparisons between groups was done by Generalized Estimation Equations at patient level. Between group changes were compared versus baseline and versus week 48 for data at week 96. Statistically significant P-values are shown in bold.
NRS: numeric rating scale,
FIHOA: functional index for hand osteoarthritis;
AUSCAN: Australian-Canadian Hand Osteoarthritis Index

TABLE 7
Clinical data from the placebo-controlled and open-label extension phase at Week 96

| | | | | P-value | P-value | P-value |
|---|---|---|---|---|---|---|
| | | | | within groups | between groups | between groups |
| Endpoint | Group | Week 96 | | vs. baseline | vs. baseline | vs. week 48 |
| Change in NRS | Denosumab | −2.3 | (−48.3) | <0.001 | 0.028 | 0.748 |
| pain (%) | Placebo | −1.3 | (−26.7) | <0.001 | — | — |
| Change in FIHOA | Denosumab | −1.9 | (−18.6) | 0.042 | 0.025 | 0.586 |
| (%) | Placebo | −0.5 | (4.9) | 0.627 | — | — |
| Change in | Denosumab | −4.9 | (−7.4) | 0.273 | 0.785 | 0.062 |
| AUSCAN (%) | Placebo | −7.4 | (−10.7) | 0.393 | — | — |
| Change in tender | Denosumab | −2.1 | (−38.0) | 0.001 | 0.579 | 0.188 |
| joint count (%) | Placebo | −2.8 | (−52.7) | 0.021 | — | — |
| Change in swollen | Denosumab | −1.2 | (28.3) | 0.003 | 0.263 | 0.366 |
| joint count (%) | Placebo | −1.8 | (−37.0) | <0.001 | — | — |
| Change in grip | Denosumab | 0.2 | (1.3) | 0.818 | 0.063 | 0.016 |
| strength (%) | Placebo | 0.6 | (3.1) | 0.569 | — | — |

Longitudinal changes compared to baseline are given and percentage changes compared to baseline. Comparisons between groups was done by Generalized Estimation Equations at patient level. Between group changes were compared versus baseline and versus week 48 for data at week 96. Statistically significant P-values are shown in bold.
NRS: numeric rating scale,
FIHOA: functional index for hand osteoarthritis;
AUSCAN: Australian-Canadian Hand Osteoarthritis Index

### Safety

Through week 48, the incidence of adverse events was higher in the placebo group versus the denosumab group (Table 8). Fourteen serious adverse events were reported during the study: seven in the denosumab group and seven in the placebo group (Table 8). Six patients discontinued the study because of an adverse event (one acute coronary event, three malignancies, one urticarial reaction, and one due to subjective intolerance to calcium/vitamin D intake). The most common adverse events were infections and musculoskeletal complaints in both groups (in denosumab: n=41 and n=24; in placebo: n=38 and n=34, respectively). Cancer occurred in three patients (all allocated to placebo). Hypocalcemia during any time in the study occurred in five patients in the denosumab group and in three patients in the placebo group (Table 9). Three events (obstipation and diverticulitis) were found to be related to the study medication (all receiving denosumab). During the extension phase, eleven new serious adverse events occurred, all not related to the study medication. Asymptomatic hypocalcemia occurred in 2 patients at week 72 (one recovered, one persisted up to week 96).

TABLE 8
Summary of safety events through week 48

| Event | Denosumab (N = 51) | Placebo (n = 49) |
|---|---|---|
| Any adverse event - no. | 98 | 125 |
| Serious adverse event - no. | 7 | 7 |
| Adverse event leading to | 3† | 3‡ |
| discontinuation - no. | | |
| Adverse event of special | | |
| interest - no. | | |
| Cancer | 0 | 3‡ |
| Infection | 41 | 39 |
| Major cardiovascular event∫ | 1 | 0 |
| Gastrointestinal event | 6 | 7 |
| Surgical and medical | 3 | 9 |
| procedures | | |
| Musculoskeletal complaints | 26 | 34 |
| Nervous system disorders | 4 | 17 |
| (incl. dizziness, vertigo, | | |
| headache) | | |
| Pulmonary and respiratory | 2 | 1 |
| complaints (noninfectious) | | |
| Rash and skin problems | 3 | 1 |
| Allergy (systemic and | 3 | 1 |
| urticaria) | | |
| Teeth problems | 3 | 3 |
| Hypocalcemia¶ | | |
| At week 12 | 2 | 0 |
| At week 24 | 1 | 1 |
| At week 36 | 2 | 1 |
| At week 48 | 3 | 1 |
| Reduced kidney function | | |
| >25% from baseline at W 12 | 2 | 3 |
| >25% from baseline at W 48∥ | 2 | 1 |

* Analyses were performed with data from the intention-to-treat population.
†In the denosumab group, one patient experienced an acute coronary syndrome, one had an urticarial skin reaction and one experienced a subjective intolerance to the calcium and vitamin D administration and discontinued the study therefore.
‡In the placebo group, two patients had breast cancer and one patient had a pancreatic adenocarcinoma with metastases and discontinued the study.
∫One patient in the denosumab group experienced an acute coronary syndrome 4 weeks after start of the study.
¶Hypocalcemia was defined as below 2.12 millimole per liter
∥Hypocalcemia at week 48 was a new finding in one patient and already present in one patient at W 12 in the denosumab group and a new finding in one patient in the placebo group.

TABLE 9
Longitudinal laboratory values

| Variable | Group | Screening | Week 12 | Week 24 | Week 36 | Week 48 |
|---|---|---|---|---|---|---|
| Calcium | Denosumab | 2.36 (0.09) | 2.38 (0.09) | 2.40 (0.12) | 2.38 (0.09) | 2.38 (0.11) |
| (mmol/liter) | | [1.97-2.55] | [2.13-2.57] | [1.83-2.67] | [2.14-2.61] | [2.10-2.61] |
| | Placebo | 2.38 (0.10) | 2.41 (0.10) | 2.38 (0.14) | 2.41 (0.11) | 2.39 (0.10) |
| | | [2.21-2.71] | [2.21-2.66] | [1.84-2.67] | [2.17-2.76] | [2.16-2.66] |
| Phosphor | Denosumab | 1.19 (0.18) | 1.14 (0.16) | — | — | 1.15 (0.18) |
| (mmol/liter) | | [0.75-1.58] | [0.76-1.52] | | | [0.71-1.57] |
| | Placebo | 1.18 (0.14) | 1.19 (0.13) | — | — | 1.17 (0.13) |
| | | [0.92-1.48] | [0.88-1.49] | | | [0.94-1.45] |
| Creatinine | Denosumab | 0.80 (0.18) | 0.82 (0.14) | — | — | 0.81 (0.15) |
| (mg/dl) | | [0.51-1.34] | [0.62-1.24] | | | [0.59-1.21] |
| | Placebo | 0.80 (0.18) | 0.85 (0.15) | — | — | 0.85 (0.17) |
| | | [0.52-1.48] | [0.58-1.23] | | | [0.62-1.27] |
| C-reactive | Denosumab | 0.33 (0.61) | 0.29 (0.44) | — | — | 0.26 (0.42) |
| protein | | [0.07-4.12] | [0.08-2.06] | | | [0.09-2.30] |
| (mg/liter) | Placebo | 0.18 (0.20) | 0.20 (0.22) | — | — | 0.38 (0.74) |
| | | [0.05-1.36] | [0.07-1.39] | | | [0.07-4.07] |

Values are mean (SD)[range]

### DISCUSSION

In this 48-week, placebo-controlled, double-blind clinical study, denosumab administered at a dose of 60 mg every 3 months reduced the radiographic erosive progression in erosive hand OA versus placebo with no significant safety signals identified. Significant effects were observed at week 24, which remained consistent and, in some cases, improved through 48 weeks of denosumab administration. Furthermore, fewer new erosive joints developed through week 48 in the denosumab group. While clinical outcome measures did not significantly change between groups in the initial 48 weeks of treatment, a significant improvement in pain and disability levels in the extension phase through week 96 was noted, suggesting that prolonged treatment with denosumab not only inhibits structural progression but also culminates in clinical improvement over time. The safety profile of denosumab was found to be comparable with previous studies and use in clinical care (22), even though the dosing regimen was doubled compared to standard regimens used in osteoporosis treatment. This is the first study demonstrating consistent benefits on radiographic progression in erosive hand OA after 24 weeks and subsequent clinical benefits after long-term treatment.

Several previous studies in erosive hand OA using therapeutic agents such as tumor necrosis factor α blocking agents (i.e., adalimumab (23, 24) and etanercept (25)), and anti-interleukin-1α and β inhibitor, lutikizumab (26), failed to show clinical efficacy in short- and long-term studies. Only one study showed some beneficial effect on structure modification, albeit in a post-hoc analysis including only the inflammatory joints (23). One recent, 6-week study with corticosteroids showed significant impact on pain in hand OA (27). Due to its short duration, any beneficial effects on structure modification and disability could not be demonstrated. The present study confirms the ability of denosumab to primarily affect radiographic progression in hand OA, thereby improving clinical status in the long-term. This points to its potential as structure-modifying drug in the treatment of erosive hand OA. Recently, a novel elective cathepsin K inhibitor demonstrated structural improvement in patients with knee OA (28). However, no benefit on pain levels was seen in this relatively short trial. These findings might create a shift in erosive hand OA treatment from pain relief toward prevention of structural or erosive damage with a cumulative impact on pain and function over time. The ultimate goal of treatment of erosive hand OA, like any other type of OA, is to avoid further joint space narrowing, cartilage degradation, and bone formation, all features of OA. By arresting radiographic damage, the burden of the disease might be substantially decreased for many patients.

It was hypothesized that the catabolic osteoclastic activity in erosive hand OA could be inhibited or even arrested by inhibiting RANKL, thereby slowing down the progression of structural damage. Similar results were seen in rheumatoid arthritis (21, 29-31), a prototypical inflammatory arthritis characterized by erosive disease. Development of erosions in RA patients was prevented using denosumab; however, denosumab administration did not impact inflammatory signs and symptoms in this treatment setting. The dosing regimen in RA was every 3 months, rather than the once every 6 months regimen used in the treatment of postmenopausal osteoporosis. The increased dosing frequency was inspired by evidence that, under chronic arthritic conditions, cellular sources of RANKL are strongly increased, extending even to other cell types such as the synovial lining layer. Because of the similarities in the impact of structural progression in erosive hand OA and RA, a similar dosing regimen was adopted for this study. However, unlike RA, there is ample evidence that erosive hand OA is not a primarily inflammatory disease (32-35). Cartilage and subchondral bone degradation appear to drive erosive hand OA progression, with inflammation being a secondary phenomenon. Therefore, other pathways may need to be targeted in order to suppress the underlying inflammation in these patients. This may explain why neither the number of swollen joints nor the sonographic outcomes for inflammation (i.e., effusion, PD signals) responded to treatment in this study. However, sonographic erosions decreased, in line with the radiographic data. Thus, despite the pathogenic differences between erosive hand OA and RA, the observations with denosumab treatment in erosive hand OA are similar to the results observed in RA.

In the current study, two validated scoring methods for structural radiographic or erosive progression were used, i.e., GUSS™ and Verbruggen and Veys anatomical phase. Both systems confirmed the reduced progression, at week 24 for the former and week 48 for the latter. The quantitative scoring system, GUSS™, was developed to demonstrate short-term changes, the benefit of which is demonstrated here. Confirming similar evolution by another scoring system (i.e., Verbruggen and Veys) may serve as an internal validation. Reliability was found to be excellent amongst the two experienced readers. Multiple clinical outcome measures were included in this study, which eventually were improved by sustained treatment with denosumab. Unfortunately, a surrogate outcome measure for disease activity in erosive hand OA is still lacking (36). Development of such a tool could facilitate clinical trial research in hand OA. Discase activity and structural progression are undeniably coupled but may be disconnected in timing. This may explain the observation that denosumab treatment inhibits structural progression as early as 6 months after therapy initiation, whereas its impact on clinical outcomes only emerges in the two year follow up. The data indicate a sustained need for RANKL inhibition in order to preserve hand function and onset of new erosive disease. In this context, the observation that the estimated odds ratio for erosive progression was 76.7% lower in the denosumab group compared to placebo in the first 48 weeks of the placebo-controlled trial supports this concept of osteoclast dependent structural damage in erosive hand OA.

No safety signals for treatment with increased interval dosing of denosumab were found in this non-osteoporotic population. A higher number of non-serious and serious adverse events were reported in the placebo group. The adverse events leading to premature discontinuation of eight patients before week 48 were not related to the study medication. Hypercalcemia was reported rarely and easily manageable before the next administration of the study drug. As expected, all BMD values increased in the denosumab group, and only at the spine in the placebo group, which might be attributed to the calcium and vitamin D administration. Inclusion of a specific subset of patients (i.e., with signs of clinical and sonographic inflammation) is both a strength and a limitation; it limits the generalizability of the results to hand OA patients with no inflammatory signs, but, on the other hand, it increases the likelihood of observing an effect due to the targeted treatment. Since hand OA is a heterogeneous disease, patient stratification may be required in clinical trials to identify hand OA patients who will benefit from specific treatments. Another limitation of this study is that it was not powered or designed to include a pre-specified statistical comparison of efficacy between the denosumab treatment arms through week 96. Finally, due to the ongoing treatment until the end of the study, safety conclusions about a potential rebound effect on bone status in this non-osteoporotic populations cannot be drawn. In summary, this placebo-controlled trial provides the first proof of concept that structural damage in the erosive type of hand OA can be modulated by a targeted therapy. Clear benefits from treatment with denosumab 60 mg every 3 months were observed in erosive hand OA patients by reducing radiographic progression and the development of new erosive joints. These improvements ultimately led to improvements in pain and disability after long-term treatment through 96 weeks.

### REFERENCES

The following references are cited in the Background and Example 1.
1. Punzi L, Ramonda R, Sfriso P. Erosive osteoarthritis. Best practice & research Clinical rheumatology. 2004; 18 (5): 739-58.
2. Kwok W Y, Kloppenburg M, Rosendaal F R, van Meurs J B, Hofman A, Bierma-Zeinstra S M. Erosive hand osteoarthritis: its prevalence and clinical impact in the general population and symptomatic hand osteoarthritis. Ann Rheum Dis. 2011; 70 (7): 1238-42.
3. Haugen I K, Englund M, Aliabadi P, Niu J, Clancy M, Kvien T K, et al. Prevalence, incidence and progression of hand osteoarthritis in the general population: the Framingham Osteoarthritis Study. Ann Rheum Dis. 2011; 70 (9): 1581-6.
4. Haugen I K, Mathiessen A, Slatkowsky-Christensen B, Magnusson K, Boyesen P, Sesseng S, et al. Synovitis and radiographic progression in non-erosive and erosive hand osteoarthritis: is erosive hand osteoarthritis a separate inflammatory phenotype? Osteoarthritis Cartilage. 2016; 24 (4): 647-54.
5. Bijsterbosch J, Watt I, Meulenbelt I, Rosendaal F R, Huizinga T W, Kloppenburg M. Clinical burden of erosive hand osteoarthritis and its relationship to nodes. Annals of the rheumatic diseases. 2010; 69 (10): 1784-8.
6. Wittoek R, Cruyssen B V, Verbruggen G. Predictors of functional impairment and pain in erosive osteoarthritis of the interphalangeal joints: comparison with controlled inflammatory arthritis. Arthritis and rheumatism. 2012; 64 (5): 1430-6.
7. Kloppenburg M, Kroon F P, Blanco F J, Doherty M, Dziedzic K S, Greibrokk E, et al. 2018 update of the EULAR recommendations for the management of hand osteoarthritis. Ann Rheum Dis. 2019; 78 (1): 16-24.
8. Haugen I K, Slatkowsky-Christensen B, Boyesen P, van der Heijde D, Kvien T K. Cross-sectional and longitudinal associations between radiographic features and measures of pain and physical function in hand osteoarthritis. Osteoarthritis and cartilage. 2013; 21 (9): 1191-8.
9. Meersseman P, Van de Vyver C, Verbruggen G, Elewaut D, Wittoek R. Clinical and radiological factors associated with erosive radiographic progression in hand osteoarthritis. Osteoarthritis and cartilage. 2015; 23 (12): 2129-33.
10. Vanhaverbeke T, Pardaens L, Wittoek R. Natural disease progression in finger osteoarthritis: results from a 10 year follow-up cohort. Scandinavian journal of rheumatology. 2020; 49 (6): 498-504.
11. Crain D C. Interphalangeal osteoarthritis. JAMA. 1961; 175:1049-53.
12. Mannoni A, Briganti M P, Di Bari M, Ferrucci L, Costanzo S, Serni U, et al. Epidemiological profile of symptomatic osteoarthritis in older adults: a population based study in Dicomano, Italy. Annals of the rheumatic diseases. 2003; 62 (6): 576-8.
13. Verbruggen G, Wittoek R, Vander Cruyssen B, Elewaut D. Morbid anatomy of ‘erosive osteoarthritis’ of the interphalangeal finger joints: an optimised scoring system to monitor disease progression in affected joints. Annals of the rheumatic diseases. 2010; 69 (5): 862-7.
14. Suda T, Takahashi N, Udagawa N, Jimi E, Gillespie M T, Martin T J. Modulation of osteoclast differentiation and function by the new members of the tumor necrosis factor receptor and ligand families. Endocrine reviews. 1999; 20 (3): 345-57.
15. Kobayashi K, Takahashi N, Jimi E, Udagawa N, Takami M, Kotake S, et al. Tumor necrosis factor alpha stimulates osteoclast differentiation by a mechanism independent of the ODF/RANKL-RANK interaction. The Journal of experimental medicine. 2000; 191 (2): 275-86.
16. Komine M, Kukita A, Kukita T, Ogata Y, Hotokebuchi T, Kohashi O. Tumor necrosis factor-alpha cooperates with receptor activator of nuclear factor kappaB ligand in generation of osteoclasts in stromal cell-depleted rat bone marrow cell culture. Bone. 2001; 28 (5): 474-83.
17. Wei S, Kitaura H, Zhou P, Ross F P, Teitelbaum S L. IL-1 mediates TNF-induced osteoclastogenesis. The Journal of clinical investigation. 2005; 115 (2): 282-90.
18. Zwerina J, Redlich K, Polzer K, Joosten L, Kronke G, Distler J, et al. TNF-induced structural joint damage is mediated by IL-1. Proceedings of the National Academy of Sciences of the United States of America. 2007; 104 (28): 11742-7.
19. Verbruggen G, Goemaere S, Veys E M. Systems to assess the progression of finger joint osteoarthritis and the effects of disease modifying osteoarthritis drugs. Clinical rheumatology. 2002; 21 (3): 231-43.
20. McClung M R, Lewiecki E M, Cohen S B, Bolognese M A, Woodson G C, Moffett A H, et al. Denosumab in postmenopausal women with low bone mineral density. The New England journal of medicine. 2006; 354 (8): 821-31.
21. So H, Cheng I T, Lau S L, Chow E, Lam T, Hung V W, et al. Effects of RANKL inhibition on promoting healing of bone erosion in rheumatoid arthritis using HR-pQCT: a 2-year, randomised, double-blind, placebo-controlled trial. Annals of the rheumatic diseases. 2021.
22. Papapoulos S, Lippuner K, Roux C, Lin C J, Kendler D L, Lewiecki E M, et al. The effect of 8 or 5 years of denosumab treatment in postmenopausal women with osteoporosis: results from the FREEDOM Extension study. Osteoporosis international: a journal established as result of cooperation between the European Foundation for Osteoporosis and the National Osteoporosis Foundation of the USA. 2015; 26 (12): 2773-83.
23. Verbruggen G, Wittoek R, Vander Cruyssen B, Elewaut D. Tumour necrosis factor blockade for the treatment of erosive osteoarthritis of the interphalangeal finger joints: a double blind, randomised trial on structure modification. Ann Rheum Dis. 2012; 71 (6): 891-8.
24. Chevalier X, Ravaud P, Maheu E, Baron G, Rialland A, Vergnaud P, et al. Adalimumab in patients with hand osteoarthritis refractory to analgesics and NSAIDs: a randomised, multicentre, double-blind, placebo-controlled trial. Annals of the rheumatic diseases. 2015; 74 (9): 1697-705.
25. Kloppenburg M, Ramonda R, Bobacz K, Kwok W Y, Elewaut D, Huizinga T W J, et al. Etanercept in patients with inflammatory hand osteoarthritis (EHOA): a multicentre, randomised, double-blind, placebo-controlled trial. Annals of the rheumatic diseases. 2018; 77 (12): 1757-64.
26. Kloppenburg M, Peterfy C, Haugen I K, Kroon F, Chen S, Wang L, et al. Phase I Ia, placebo-controlled, randomised study of lutikizumab, an anti-interleukin-1 alpha and anti-interleukin-1beta dual variable domain immunoglobulin, in patients with erosive hand osteoarthritis. Annals of the rheumatic diseases. 2019; 78 (3): 413-20.
27. Kroon F P B, Kortekaas M C, Boonen A, Bohringer S, Reijnierse M, Rosendaal F R, et al. Results of a 6-week treatment with 10 mg prednisolone in patients with hand osteoarthritis (HOPE): a double-blind, randomised, placebo-controlled trial. Lancet (London, England). 2019; 394 (10213): 1993-2001.
28. Schieker M, Conaghan P G, Mindeholm L, Praestgaard J, Solomon D H, Scotti C, et al. Effects of Interleukin-1beta Inhibition on Incident Hip and Knee Replacement: Exploratory Analyses From a Randomized, Double-Blind, Placebo-Controlled Trial. Annals of internal medicine. 2020; 173 (7): 509-15.
29. Cohen S B, Dore R K, Lane N E, Ory P A, Peterfy C G, Sharp J T, et al. Denosumab treatment effects on structural damage, bone mineral density, and bone turnover in rheumatoid arthritis: a twelve-month, multicenter, randomized, double-blind, placebo-controlled, phase II clinical trial. Arthritis and rheumatism. 2008; 58 (5): 1299-309.
30. Takeuchi T, Tanaka Y, Ishiguro N, Yamanaka H, Yoneda T, Ohira T, et al. Effect of denosumab on Japanese patients with rheumatoid arthritis: a dose-response study of AMG 162 (Denosumab) in patients with Rheumatoid arthritis on methotrexate to Validate inhibitory effect on bone Erosion (DRIVE)-a 12-month, multicentre, randomised, double-blind, placebo-controlled, phase II clinical trial. Annals of the rheumatic diseases. 2016; 75 (6): 983-90.
31. Takeuchi T, Tanaka Y, Soen S, Yamanaka H, Yoneda T, Tanaka S, et al. Effects of the anti-RANKL antibody denosumab on joint structural damage in patients with rheumatoid arthritis treated with conventional synthetic disease-modifying antirheumatic drugs (DESIRABLE study): a randomised, double-blind, placebo-controlled phase 3 trial. Annals of the rheumatic diseases. 2019; 78 (7): 899-907.
32. Wittoek R, Jans L, Lambrecht V, Carron P, Verstraete K, Verbruggen G. Reliability and construct validity of ultrasonography of soft tissue and destructive changes in erosive osteoarthritis of the interphalangeal finger joints: a comparison with MRI. Ann Rheum Dis. 2011; 70 (2): 278-83.
33. Kortekaas M C, Kwok W Y, Reijnierse M, Huizinga T W, Kloppenburg M. In erosive hand osteoarthritis more inflammatory signs on ultrasound are found than in the rest of hand osteoarthritis. Ann Rheum Dis. 2013; 72 (6): 930-4.
34. Haugen I K, Boyesen P, Slatkowsky-Christensen B, Sesseng S, Bijsterbosch J, van der Heijde D, et al. Comparison of features by MRI and radiographs of the interphalangcal finger joints in patients with hand osteoarthritis. Ann Rheum Dis. 2012; 71 (3): 345-50.
35. Damman W, Liu R, Bloem J L, Rosendaal F R, Reijnierse M, Kloppenburg M. Bone marrow lesions and synovitis on MRI associate with radiographic progression after 2 years in hand osteoarthritis. Ann Rheum Dis. 2017; 76 (1): 214-7.
36. Wittoek R, Kroon F P B, Kundakci B, Abhishek A, Haugen I K, Berenbaum F, et al. Report from the Hand Osteoarthritis Working Group at OMERACT 2018: Update on Core Instrument Set Development. The Journal of rheumatology. 2019; 46 (9): 1183-7.

### Example 2. Rationale for 45 mg Q3M Dosing

Prolia® (denosumab) has been approved for the treatment of post-menopausal osteoporosis (PMO). The recommended dose is 60 mg Q6M (every six months). In Example 1, denosumab was instead administered at 60 mg Q3M. This dosing was selected in view of earlier evidence, including data for certain phase 2 Rheumatoid Arthritis (RA) studies. Higher dose or shorter interval dosing regimens showed earlier effects or a trend toward more inhibition of bone destruction (37, 38). In a Japanese study in RA patients, effects on erosion score were achieved with denosumab 60 mg dosed every 2 or 3 months but not with 6 months dosing (38). It is believed that RANKL levels in inflammatory conditions are higher than RANKL levels in osteoporosis patients. Therefore, the denosumab dose used in osteoporosis treatment (60 mg Q6M) may not provide sufficient osteoclast suppression. The dose in Example 1 (60 mg Q3M) was thus chosen to ensure osteoclast suppression.

Patients with active RA and patients with hand erosive osteoarthritis may also differ. RA patients could potentially have higher RANKL levels because the inflamed joints are a rich source of RANKL. Thus, as compared to PMO and RA, different denosumab doses may be desirable to reduce bone erosions. In a study by Cohen et al. (37), CTx % change from baseline was measured at 3, 6, and 12 months in subjects receiving 60 mg Q6M or 180 mg Q6M. The 3 months suppression of CTx (a marker of osteoclast activity) was similar between these 2 groups (−73%; Table 2 of Cohen et al.) of RA patients, suggesting that not all of the denosumab 60 mg dose was consumed by month 3.

The inventors believe that RANKL levels in patients with erosive hand osteoarthritis may not be as high as in RA patients; therefore, there is a rationale to use a dose of 45 mg Q3M instead of 60 mg Q3M. By decreasing the dose to 45 mg Q3M, it is believed that the dosing regimen will maintain the benefits of denosumab while decreasing the risk for potential adverse events.

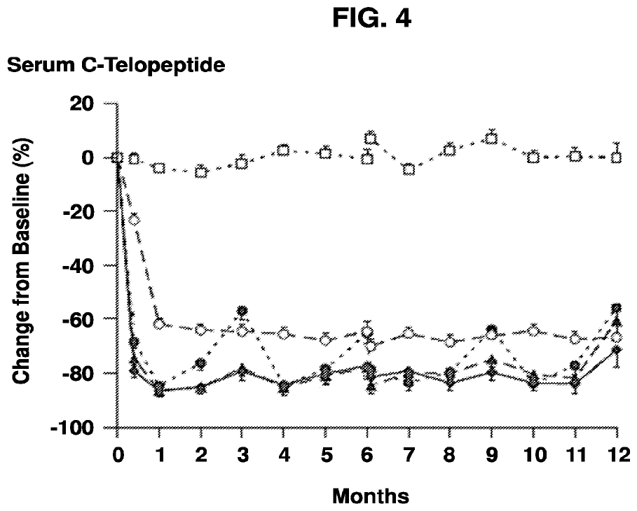

This is further supported by CTx level. FIG. 4 (see also reference 39, FIG. 2E) depicts the serum CTx levels in subjects with osteoporosis receiving placebo (□), denosumab 6 mg (●), 14 mg (Δ), 30 mg (⋄) every 3 months. A denosumab 30 mg Q3M dose was able to keep CTx suppressed in osteoporosis patients. In patients with hand erosive OA, a slightly higher dose will likely be needed to keep CTx suppressed during the 3 months dosing interval and justifies 45 mg Q3M dosing.

### REFERENCES

The following references are cited in Example 2.
37. Cohen et al., ARTHRITIS & RHEUMATISM Vol. 58, No. 5, May 2008, pp 1299-1309.
38. Ishiguro et al., Rheumatology, Volume 58, Issue 6, June 2019, Pages 997-1005.
39. McClung M. et al., (2006) N Engl J Med; 354:821-31.

All references, including publications, patent applications, and patents, cited herein are hereby incorporated by reference to the same extent as if each reference were individually and specifically indicated to be incorporated by reference and were set forth in its entirety herein.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the present disclosure and does not pose a limitation on the scope of the disclosure unless otherwise claimed. No language in the specification should be construed as indicating any non-claimed element as essential to the practice of the disclosure.

Example embodiments of this disclosure are described herein, including the best mode known to the inventors for carrying out the disclosure. Variations of the example embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A method of treating erosive hand osteoarthritis (OA) in a subject in need thereof, comprising administering to the subject denosumab in an amount equivalent to about 45 mg to about 60 mg denosumab administered once every about 3 months.

2. A method of reducing radiographic erosive progression, joint space narrowing, cartilage degradation, and/or bone formation in a subject in need thereof, comprising administering to the subject denosumab in an amount equivalent to about 45 mg to about 60 mg denosumab administered once every about 3 months.

3. A method of inhibiting the development of new erosive joints in a subject in need thereof, comprising administering to the subject denosumab in an amount equivalent to about 45 mg to about 60 mg denosumab administered once every about 3 months.

4. The method according to claim 2 or 3, wherein the subject has been diagnosed with erosive hand osteoarthritis (OA).

5. A method of reducing pain in a subject with erosive hand osteoarthritis (OA), comprising administering to the subject denosumab in an amount equivalent to about 45 mg to about 60 mg denosumab administered once every about 3 months.

6. The method according to any one of claims 1 to 5, wherein the administration reduces pain in the subject as assessed by numeric rating scale (NRS) pain.

7. The method according to any one of claims 1 to 6, wherein the administration inhibits the development of new erosive IP joints in the subject and/or inhibits the development of ‘E’ phases in ‘S/J’ IP joints in the subject.

8. The method according to any one of claims 1 to 7, wherein the administration reduces the number of ‘S/J’ IP joints that develop ‘E’ phases in the subject.

9. The method according to any one of claims 1 to 8, wherein the administration improves erosive IP finger joint remodeling in the subject.

10. The method according to any one of claims 1 to 9, wherein the administration increases total GUSS™ in the subject.

11. The method according to any one of claims 1 to 10, wherein the administration improves joint function in the subject.

12. The method according to any one of claims 1 to 11, wherein:
the subject does not exhibit a dose limiting toxicity (DLT) during denosumab administration; and/or
the subject does not exhibit any grade 3 or grade 4 adverse events associated with denosumab during denosumab administration.

13. The method according to any one of claims 1 to 12, wherein the subject is not hypocalcemic immediately prior to or during denosumab administration.

14. The method according to any one of claims 1 to 13, comprising administering to the subject denosumab in an amount equivalent to about 45 mg to less than 60 mg denosumab administered once every about 3 months.

15. The method according to any one of claims 1 to 13, comprising administering to the subject about 45 mg denosumab once every about 3 months.

16. The method according to any one of claims 1 to 13, comprising administering to the subject about 60 mg denosumab once every about 3 months.

17. The method according to any one of claims 1 to 13, wherein the amount of denosumab provides:
a. a mean serum concentration of denosumab substantially similar to that achieved by administering 45 mg denosumab once every about 3 months; and/or
b. a mean plasma area under the curve (AUC0-∞) substantially similar to that achieved by administering 45 mg denosumab once every about 3 months; and/or
c. a difference between the mean plasma Cmax at steady state and a mean plasma Cmin at steady state substantially similar to that achieved by administering 45 mg denosumab once every about 3 months; and/or
d. a mean plasma Cmax at steady state substantially similar to that achieved by administering denosumab 45 mg once every about 3 months.

18. The method according to any one of claims 1 to 13, wherein the amount of denosumab provides:
a. a mean serum concentration of denosumab substantially similar to that achieved by administering 60 mg denosumab once every about 3 months; and/or
b. a mean plasma area under the curve (AUC_{0-∞}) substantially similar to that achieved by administering 60 mg denosumab once every about 3 months; and/or
c. a difference between the mean plasma Cₘₐₓ at steady state and a mean plasma Cₘᵢₙ at steady state substantially similar to that achieved by administering 60 mg denosumab once every about 3 months; and/or
d. a mean plasma Cₘₐₓ at steady state substantially similar to that achieved by administering denosumab 60 mg once every about 3 months.

19. The method according to any one of claims 1 to 18, wherein the subject is administered denosumab for at least 12 weeks.

20. The method according to any one of claims 1 to 19, wherein the subject is administered denosumab for at least 48 weeks.

21. The method according to any one of claims 1 to 20, wherein the subject is administered denosumab for at least 96 weeks.

22. The method according to any one of claims 1 to 21, wherein the subject is administered denosumab once every about 3 months for at least 2 cycles.

23. The method according to any one of claims 1 to 22, wherein denosumab is administered to the subject by injection.

24. The method according to any one of claims 1 to 23, wherein the method further comprises administering to the subject at least one therapeutic agent chosen from analgesics and non-steroidal anti-inflammatory drugs.

25. The method according to any one of claims 1 to 24, wherein the subject exhibits at least one sign of clinical or sonographic inflammation prior to denosumab administration.

26. The method according to any one of claims 1 to 25, wherein the subject has not been diagnosed with vitamin D deficiency, a chronic inflammatory rheumatic disease, psoriasis, cancer, or a chronic infectious disease.

27. The method according to any one of claims 1 to 26, wherein the subject has not been previously treated for erosive hand osteoarthritis.

28. The method according to any one of claims 1 to 26, wherein the subject was previously treated for erosive hand osteoarthritis.

29. The method according to claim 28, wherein the subject was previously treated with at least one therapeutic agent chosen from tumor necrosis factor α (TNFα) blocking agents, anti-interleukin-1α inhibitors, anti-interleukin-1β inhibitors, and combinations thereof.

30. The method according to claim 28 or 29, wherein the subject was previously treated with at least one therapeutic agent chosen from adalimumab, etanercept, lutikizumab, and combinations thereof.
